(19) United States
(12) Patent Application Publication
(10) Pub. No.: US 2022/0142566 A1
(43) Pub. Date: May 12, 2022

(54) CLOSED-LOOP DETECTION AND TREATMENT OF RADIATION AND TOXIC AGENTS

(71) Applicant: Massachusetts Institute of Technology, Cambridge, MA (US)

(72) Inventors: Hen-Wei Huang, Cambridge, MA (US); James Byrne, Boston, MA (US); Carlo Giovanni Traverso, Newton, MA (US)

(73) Assignee: Massachusetts Institute of Technology, Cambridge, MA (US)

(21) Appl. No.: 17/470,942

(22) Filed: Sep. 9, 2021

Related U.S. Application Data
(60) Provisional application No. 63/076,147, filed on Sep. 9, 2020.

(51) Int. Cl. A61B 5/00; A61B 6/00; A61B 5/1473; A61K 31/4748; A61M 31/00; A61M 5/158

(52) U.S. Cl. CPC A61B 5/4839; A61B 6/425; A61B 6/4258; A61B 5/1473; A61B 5/4845; A61B 5/6861; A61M 2205/21; A61M 31/00; A61M 5/158; A61M 2210/1053; A61M 2205/3303; A61M 2230/20; A61K 31/4748

(57) ABSTRACT

Articles, systems, and methods for rapid administration and active pharmaceutical compositions to subjects exposed to radiation and/or toxins are provided by this disclosure. There are currently few interventional technologies to protect against long-term morbidity and mortality from exposure to radiation and chemical warfare. Furthermore, as commercially available technologies such as additive manufacturing and small-scale chemical reactors have become more prevalent, the risk of misuse of such technologies by terrorists and rogue nation states to manufacture nuclear, radiologic, and chemical weapons continues to increase. Articles, systems, and methods described herein combine sensing technologies with drug release components, in order to provide an approach for rapid diagnosis of and response to exposure to radiation and toxic agents.

### RELATED APPLICATIONS

This application claims priority under 35 U.S.C. § 119(e) to U.S. Provisional Application No. 63/076,147, filed Sep. 9, 2020, and entitled “Closed-Loop Ingestible Robot for Radiation and Chemical Sensing,” which is incorporated herein by reference in its entirety for all purposes.

### GOVERNMENT SPONSORSHIP

This invention was made with Government support under Grant No. FA8650-21-2-7120 awarded by the Defense Advanced Research Projects Agency (DARPA). The Government has certain rights in the invention.

### TECHNICAL FIELD

Articles, systems, and methods for rapid administration of active pharmaceutical compositions to subjects exposed to radiation and/or toxins are generally described.

### BACKGROUND

The modern risk of nuclear and chemical terrorism continues to increase as technological advances to support such capability are globally disseminated. Furthermore, nuclear and chemical terrorism can take several forms, such as forceful takeover of a nuclear power facility, targeting of a country's nuclear power facilities by terrorists or rogue states, intentional detonation of a nuclear weapon, or the use of radiologic dispersion or exposure articles. Radiation and chemical exposure from one of these events can result in systemic toxicity on multiple biologic systems depending on the type of radioactive or chemical particle, total dose, and the degree of tissue penetration. For example, radiation doses of 2 Gray (Gy) or more can cause acute radiation syndrome, where the most immediate problems are bone marrow failure and gastrointestinal (GI) damage. Current medical management relies on a limited arsenal of therapeutic strategies whose efficacy depreciates with increased radiation exposure and time to treat, emphasizing the current need for improved monitoring and treatment paradigms.

### SUMMARY

Articles, systems, and methods described herein combine sensing technologies with drug release components, in order to provide an approach for rapid diagnosis of and response to exposure to radiation and toxic agents. The subject matter of the present invention involves, in some cases, interrelated products, alternative solutions to a particular problem, and/or a plurality of different uses of one or more systems and/or articles.

In one aspect, an article is provided. According to certain embodiments, the article has a capacity for ingestion, an ability to detect and quantify radiation or one or more chemicals, and deliver a discrete dose of a therapeutic agent.

In one aspect, an article is provided. According to certain embodiments, the article comprises: one or more sensors to detect radiation or a chemical; a drug delivery mechanism; and an ingestible structure capable of gastric residence and supporting the one or more sensors and the drug delivery mechanism; wherein the drug delivery mechanism delivers a therapeutic agent related to the detected radiation or chemical into a gastrointestinal tract in response to a threshold level of detection by the one or more sensors.

In one aspect, an article is provided. According to certain embodiments, the article comprises: a central core, a tissue-engaging surface, a plurality of arms connected to the central core, one or more radiation sensors on the plurality of arms, and a drug releasing component associated with the tissue-engaging surface.

In one aspect, an article is provided. According to certain embodiments, the article comprises: one or more sensors, including at least one radiation sensor; and a drug releasing component; wherein: the article is an ingestible structure capable of gastric residence wherein the drug releasing component is configured to deliver a counter-radiation agent into a gastrointestinal tract using the drug release component, in response to a threshold level of detection by the radiation sensor.

In one aspect, a method is provided. According to certain embodiments, the method comprises: detecting radiation using a radiation sensor of an article located within a subject; and upon detection of radiation above a threshold level, administering an active pharmaceutical agent to the subject using the article.

In one aspect, a self-righting article for the detection of radiation is provided. According to certain embodiments, the self-righting article for the detection of radiation comprises: a central core comprising a tissue-engaging surface; a tissue interfacing component associated with the tissue-engaging surface; a radiation sensor configured to detect radiation; three or more deployable arms have a retracted configuration and an expanded configuration, the retracted configuration comprising the three or more deployable arms located proximate the central core, and the expanded configuration comprising the three or more deployable arms located away from the central core, the three or more deployable arms configured to change configuration upon detection of radiation above a threshold value by the radiation sensor; wherein changing configuration from the retracted configuration to the expanded configuration orients the self-righting article such that the tissue-engaging surface contacts a tissue of a subject at a location internal to the subject.

In one aspect, an article is provided. According to certain embodiments, the article comprises: a central core, a tissue-engaging surface, a plurality of arms connected to the central core, one or more chemical sensors on the plurality of arms, wherein the one or more sensors are configured to detect a toxic chemical, and a drug releasing component associated with the tissue-engaging surface.

In one aspect, an article is provided. According to certain embodiments, the article comprises: one or more sensors, including at least one chemical sensor configured to detect a toxin; and a drug releasing component; wherein: the article is an ingestible structure capable of gastric residence wherein the drug releasing component is configured to deliver a counter-toxin into a gastrointestinal tract using the drug release component, in response to a threshold level of detection by the chemical sensor.

In one aspect, a method is provided. According to certain embodiments, the method comprises: detecting a toxin using a chemical sensor of an article located within a subject; and upon detection of the toxin, administering an active pharmaceutical agent to the subject using the article.

In one aspect, a self-righting article for the detection of radiation is provided. According to certain embodiments, the self-righting article for the detection of radiation comprises: a central core comprising a tissue-engaging surface; a tissue interfacing component associated with the tissue-engaging surface; a chemical sensor configured to detect a toxin; three or more deployable arms have a retracted configuration and an expanded configuration, the retracted configuration comprising the three or more deployable arms located proximate the central core, and the expanded configuration comprising the three or more deployable arms located away from the central core, the three or more deployable arms configured to change configuration upon the detection of the toxin by the chemical sensor; wherein changing configuration from the retracted configuration to the expanded configuration orients the self-righting article such that the tissue-engaging surface contacts a tissue of a subject at a location internal to the subject.

Other advantages and novel features of the present invention will become apparent from the following detailed description of various non-limiting embodiments of the invention when considered in conjunction with the accompanying figures. In cases where the present specification and a document incorporated by reference include conflicting and/or inconsistent disclosure, the present specification shall control.

### BRIEF DESCRIPTION OF THE DRAWINGS

Non-limiting embodiments of the present invention will be described by way of example with reference to the accompanying figures, which are schematic and are not intended to be drawn to scale unless otherwise indicated. In the figures, each identical or nearly identical component illustrated is typically represented by a single numeral. For purposes of clarity, not every component is labeled in every figure, nor is every component of each embodiment of the invention shown where illustration is not necessary to allow those of ordinary skill in the art to understand the invention. In the figures:

FIGS. 1A-1C present perspective schematic illustrations of an exemplary article comprising a central core and a plurality of arms, according to certain embodiments;

FIGS. 2A-2C present perspective schematic illustrations of an exemplary article comprising a central core, a tissue-engaging surface, and a plurality of arms, according to certain embodiments;

FIGS. 3A-3D present cross-sectional schematic illustrations of an exemplary article comprising a central core and a plurality of arms in the process self-orienting, according to certain embodiments;

FIG. 4 presents an exemplary method for orienting articles comprising active pharmaceutical agents, and using them for delivery, in some embodiments;

FIGS. 5A-5C present cross-sectional schematic illustrations of an exemplary article transitioning from a first configuration to a second configuration, according to certain embodiments;

FIGS. 6A-6B present cross-sectional schematic illustrations of an exemplary drug release component, according to certain embodiments;

FIG. 7 presents a cross-sectional schematic illustration of an exemplary measurement system for removal force, according to certain embodiments;

FIG. 8 presents an exploded-perspective schematic illustration of an exemplary article, according to certain embodiments;

FIGS. 9A presents an illustration of an exemplary circuit of an article, according to certain embodiments;

FIGS. 9B-9D present photographs of an exemplary circuit of an article, according to certain embodiments;

FIGS. 10A-10C present schematic, perspective illustrations of an article comprising radiation sensors, according to certain embodiments;

FIG. 11 presents an exemplary system architecture of an article, according to certain embodiments;

FIGS. 12A-12B present photographs of an exemplary article and circuit, according to certain embodiments;

FIG. 13 presents cross-sectional schematic illustrations of an exemplary article, according to certain embodiments;

FIG. 14 presents exemplary measurements from a radiation sensor of an exemplary article, according to certain embodiments;

FIGS. 15A-15C present photographs of exemplary drug delivery articles, according to certain embodiments;

FIGS. 16A-16B present photographs of exemplary drug delivery articles, according to certain embodiments;

FIG. 17 presents measurements of white blood cell counts of subjects upon delivery of sargramostim via various exemplary delivery methods, according to certain embodiments; and

FIG. 18 presents measurements of heart rates of subjects upon delivery of atropine via various exemplary delivery methods, according to certain embodiments.

### DETAILED DESCRIPTION

Articles, systems, and methods for rapid administration of active pharmaceutical compositions to subjects exposed to radiation and/or toxins are generally provided. There are currently few interventional technologies to protect against long-term morbidity and mortality from exposure to radiation and chemical warfare. Furthermore, as commercially available technologies such as additive manufacturing and small-scale chemical reactors have become more prevalent, the risk of misuse of such technologies by terrorists and rogue nation states to manufacture nuclear, radiologic, and chemical weapons continues to increase. Articles, systems, and methods described herein generally relate to ingestible, closed-loop, sensing technologies further comprising drug release components, in order to provide an approach for rapid diagnosis of and response to exposure to radiation and toxic agents.

In one aspect, articles suitable for delivering active pharmaceutical agents to subjects are provided. These articles may be configured to deliver counter-radiation agents, e.g., in response to exposure to ionizing radiation. In an exemplary embodiment, an ingestible article capable of sensing and quantifying radiation and/or chemical exposure and capable of delivery of a solid dosage form is provided. In some embodiments, the article has a Kirigami (or Kirigami-like) design configured for relatively long gastric residence (e.g., greater than or equal to 24 hours), as well as self-orientation to the gastric wall. In some embodiments, upon exposure to a threshold dose of radiation or chemical exposure, the article actuates a solid drug needle into the gastric wall (e.g., for therapeutic purposes in response to the radiation or chemical exposure). The article is configured to detect one or more forms of radiation, numerous forms of chemical exposure, and any combination of one or more of these.

Articles described herein, for example, may have one or more of the following: 1) a design that is ingestible through a capsule; 2) one or multiple radiation or chemical sensors; 3) a structure capable of short or long gastric residence; and/or 4) a configuration allowing closed-loop sensing to result in actuation of a drug needle, or other drug delivery mechanism, delivered into the GI tract.

To address such threats or risks, a closed-loop surveillance and drug delivery platform for sensitive radiation and/or chemical monitoring and rapid therapeutic benefit is provided. This platform combines point-of-exposure drug delivery with ingestible radiation and chemical sensing technology, allowing monitoring of internal contamination of the body's tissues for a more precise dosing strategy and immediate treatment, thereby hopefully decreasing morbidity and mortality.

Given the types of radiation or chemicals that could be encountered, in the context of the present disclosure, it has been recognized that an ingestible system is desirable to appropriately assess radiation or chemical dose to critical radiation-sensitive organs, including bone marrow and the intestines. It has been recognized herein that, for soldiers and first aid responders at risk for radiation and chemical exposure, delayed treatment may result in serious morbidity and mortality.

In some embodiments, the article has a particular configuration including a particular size and/or shape such that the article can be administered to a subject (e.g., orally). In some embodiments, the article is configured to adopt a shape and/or size in vivo that slows or prevents further transit in a body (e.g., gastric) cavity (e.g., passage from the body of the stomach through the pylorus). In some embodiments, the article adopts a shape and/or size configured for retention (e.g., gastric residence). In some embodiments, the article adopts the shape and/or size for retention upon release from a soluble capsule/container and/or soluble retaining structure/element. In some embodiments, the article is configured for adopting a shape and/or size configured for gastric residence after being stored in its encapsulated shape and/or size for durations greater than 24 hours, including up to about one year. In some embodiments, the mechanical properties of the article are optimized for safe transient retention of all or a portion of the article in an internal cavity such as the gastric cavity for durations greater than 24 hours, including up to about one year.

Certain embodiments of structures and systems described herein may offer certain advantages as compared to traditional compositions and structures and systems configured for internal retention and/or drug release, for example, in their ability to adopt a shape and/or size small enough to be ingested by a subject; adopt a shape and/or size internally that slows or prevents further transit in a body cavity (e.g., the gastric cavity) (e.g., passage from the body of the stomach through the pylorus;) be loaded at high levels (e.g., high mass fraction) with therapeutic, diagnostic, and/or enhancement agents; facilitate controlled release of such therapeutic, diagnostic, and/or enhancement agents (e.g., upon detection of a signal from a sensor including, for example, radiation and/or chemical sensing); maintain activity/stability of such therapeutic, diagnostic, and/or enhancement agents in a hostile environment such as the gastric environment for an extended duration; maintain safety with low to no potential for gastric or intestinal obstruction and/or perforation; and/or degrade/dissolve/disassociate into one or more forms configured for passing through a gastrointestinal tract. In certain embodiments, the articles and systems described herein can be configured with durable residence times greater than at least twenty-four hours and lasting up to about one year, or more. In some embodiments, the systems, articles, and methods described herein are compatible with subjects, including, but not limited to, humans and non-human animals.

The articles and systems described herein may be modular/multi-component (i.e., formed of multiple interconnected subcomponents.) In some embodiments, the article comprises one or more sensors, three or more arms, a central core, and/or a drug release component.

In some embodiments, the article is configured for adopting a shape and/or size configured for gastric deployment (after being stored in its encapsulated/folded shape and/or size) for the residence time period. In some embodiments, the residence time period is greater than or equal to 24 hours, greater than or equal to 48 hours, greater than or equal to 3 days, greater than or equal to 7 days, greater than or equal to 1 month, greater than or equal to 6 months, or greater than or equal to 1 year. In certain embodiments, the residence time period is less than or equal to 2 years, less than or equal to 1 year, less than or equal to 6 months, less than or equal to 1 month, less than or equal to 7 days, less than or equal to 3 days, or less than or equal to 48 hours. Combinations of the above-referenced ranges are also possible (e.g., greater than or equal to 24 hours and less than or equal to 2 years, greater than or equal to 24 hours and less than or equal to 1 year, greater than or equal to 48 hours and less than or equal to 7 days, greater than or equal to 3 days and less than or equal to 1 month, greater than or equal to 7 days and less than or equal to 6 months, greater than or equal to 1 month and less than or equal to 1 year). Other ranges are also possible.

Advantageously, the structures and components described herein are configured to provide closed-loop monitoring and detection of radiation and/or chemical compounds e.g., via one or more sensors at a location internal to a subject without the need for a surgical procedure (e.g., an incision, implantation within layers of tissue). In some cases, the article may be administered to a subject. In some embodiments, the article is administered orally, rectally, vaginally, nasally, or uretherally. In some embodiments, the article is contained within a containing structure (e.g., during administration) and, upon reaching the location internal to the subject (e.g., in the gastrointestinal tract), at least a portion of the containing structure degrades such that the article obtains a configuration configured for gastric residence. In some embodiments, the article is administered directly to a subject (e.g., without a containing structure).

Advantageously, in some embodiments, the articles described herein are configured for rapid re-configuration (e.g., re-orientation) and delivery of a therapeutic agent in response to radiation and/or chemical toxin to which the subject has been exposed.

In some embodiments, an article includes a central core and a plurality of arms attached to and configured to rotate relative to the central core. In some embodiments, the central core and/or distal portions of the arms may be equipped with drug release components (sometimes referred to herein as a tissue-interfacing component, e.g., that may comprise needles) configured to deliver an active pharmaceutical compound to the esophagus, or other structure, without perforation. However, embodiments without drug release components, (e.g., embodiments generally intended for data collection and/or monitoring) are also envisioned. The article may be able to fold into a retracted form that can be easily delivered to the esophagus or other anatomical structure in any appropriate manner. Upon reaching the esophagus, or other desired location within a subject's body, the article may be permitted to transform from the retracted configuration into an expanded configuration in which the arms of the article may pivot away from the central body. In some embodiments, this results in penetration of the drug release component (e.g., the penetration of a needle of the drug release component) into the esophageal mucosa and delivery of an active pharmaceutical compound to the esophageal mucosa. However, in some embodiments, the expanded configuration may be used principally for improved gastric retention without injection. Penetration of the drug release components into the esophageal mucosa and subsequent delivery of the active pharmaceutical compound may occur immediately upon transformation into the expanded configuration. However, in some embodiments, penetration of the drug release component instead occurs in response to external signals or conditions. For example, in some embodiments, the drug release component is configured to penetrate a subject in response to exposure of the article to radiation exceeding a threshold level.

In some embodiments, the tissue-interfacing component comprises a needle, a biopsy component, a projectile, a plurality of microneedles, a hook, a mucoadhesive patch, or combinations thereof. In certain embodiments, as described herein and above, the tissue interfacing component is configured to penetrate tissue (e.g., skin, tongue, tissue of the GI tract such as GI mucosal tissue). In some embodiments, the tissue in penetrated with a force of greater than or equal to 1 mN and less than or equal to 20 N (e.g., greater than or equal to 10 mN and less than or equal to 20 mN, greater than or equal to 1 mN and less than or equal to 100 mN, greater than or equal to 20 mN and less than or equal to 1 N, greater than or equal to 1 N and less than or equal to 20 N, greater than or equal to 10 N and less than or equal to 20 N).

In response to an external signal or condition, the article may reconfigure from the expanded configuration with a larger transverse dimension (e.g., a width or diameter) to the retracted configuration with a smaller transverse dimension to enable the article to subsequently pass through the gastrointestinal tract or to otherwise be removed from the esophagus or other anatomical structure.

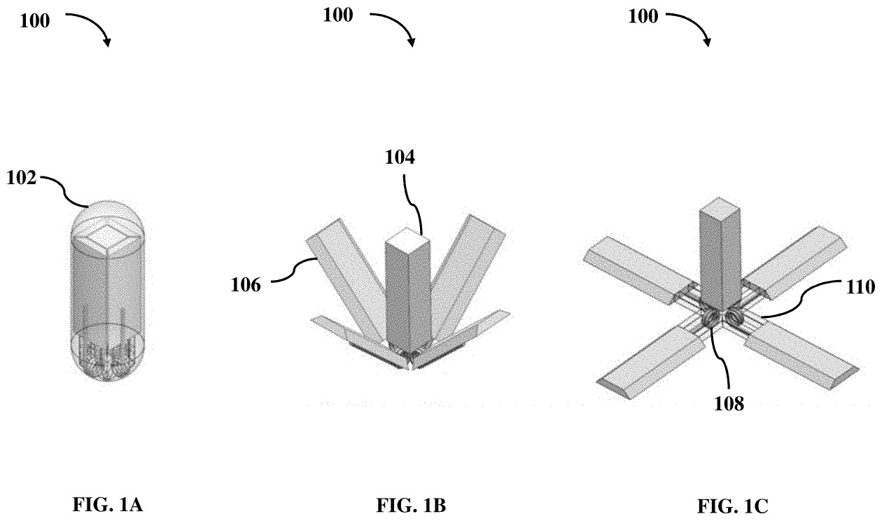

FIGS. 1A-1C depict one embodiment of an article 100 in various configurations. FIG. 1A shows the article in a retracted configuration within a capsule 102. FIG. 1B shows the article in a partially expanded configuration, while FIG. 1C shows the article in a fully expanded configuration. In some embodiments, an article may include a central core and a plurality of arms that are rotatably coupled to a central core. In some embodiments, the plurality of arms comprises two, three, four, five, six, eight, ten, or more arms. In some embodiments, the arms may be biased away from the central core via one or more flexible elastic components. For example, referring to the exemplary embodiment of FIGS. 1A-1C, article 100 includes central core 104 and plurality of arms 106. Depending on the particular embodiment, a flexible elastic component may be attached to and extend between an associated arm and the central core and/or between adjacent arms. For example, in FIGS. 1A-1C, the arms 106 may be coupled to one another with one or more elastic components, such as beams 110. In either case, elastic energy stored in a flexible elastic component when the article is in a retracted configuration may apply a force to the associated arms to bias the article from the retracted configuration to the expanded configuration during deployment. Thus, the arms may be biased radially outward from the body by the associated flexible elastic components.

Specifically, as shown in FIGS. 1A-1C, the beams may have a L shape, or other angled shape, such that the straight portions of the beam are aligned with and extend along at least a portion of a length of adjacent arms. The beams, or other elastic components, may be attached to the arms using any appropriate method including adhesives, welding, mechanical interlocking features, interference fits and/or any other appropriate attachment method. While the use of flexible elastic beams is described in relation to the figures, other appropriate flexible elastic components that may be used include but are not limited to springs (such as torsion springs) connected between an arm and central core, elastic living hinges disposed between an arm and central core, and/or any other flexible elastic structure configured to bias the arms away from the central core. Accordingly, it should be understood that various components and configurations may be used to apply the desired deployment forces to an article.

In some embodiments, an article may also include one or more biasing components that, when signaled, apply a force to the arms to bias the arms towards a central core of the article such that the article is biased from the expanded configuration to the retracted configuration. Biasing components, such as actuated springs 108 of FIGS. 1A-1C, may couple the arms to the central core. In embodiments such as that presented in FIGS. 1A-1C, the actuated springs 108 are torsional springs, which including one or more coils with the opposing ends attached to the core and a corresponding arm as shown in the figure. The spring, or other biasing component, may be attached to the core and arms using any appropriate attachment method similar to those noted above for the elastic component. The force applied by the biasing components may be sufficient to overcome a force applied by the elastic beams, or other elastic component, that is applied in an opposing direction, to apply an overall force that biases the article into the retracted configuration. In some embodiments, the biasing components may apply a force to the arms to bias the article from the expanded configuration to the retracted configuration without heating. For example, deformation of the biasing components may result in a restoring force that biases the arms towards the core.

According to certain embodiments, an article in the expanded configuration may be retained in a location internal to a subject such as the gastrointestinal tract of the subject, relative to an article in the retracted configuration. In other words, in some embodiments, the article may be placed in the expanded configuration to facilitate long-term gastric retention of the article. According to certain embodiments, the article can remain in a stomach until signaled to exit the stomach. In some embodiments, an article in the expanded configuration can be placed in an injection mode, wherein the arms remain in an expanded configuration, but their position is adjusted to better stabilize the capsule upon injection.

The phrase “location internal to a subject” as used herein generally refers to an internal cavity (e.g., the mouth, the esophagus, the small intestine, the colon, the duodenum, the ileum, the jejunum, the stomach, or the rectum) of the subject. In some embodiments, the location internal to the subject is proximate (e.g., adjacent, directly adjacent) a gastric orifice such as the pylorus. In some embodiments, the article is configured to reside adjacent the gastric orifice such as the pylorus (e.g., the article has a largest cross-sectional area which does not permit passage through the pylorus). Those of ordinary skill in the art would understand, based upon the teachings of this specification, that an article is retained at a location internal to a subject when it does not substantially transit from said location absent a physical, chemical, or mechanical change to the article. By way of example and without wishing to be bound by a literal interpretation of such, an article is retained a location internal to a subject when it remains substantially proximate (e.g., adjacent, in contact with) that location over the duration of a residence time period (e.g., greater than or equal to 24 hours). By contrast, by way of a comparative example and without wishing to be bound by a literal interpretation of such, an article is not considered retained at a location internal to a subject as it transits the gastrointestinal tract (e.g., driven by gastrointestinal forces and/or motion such that it moves through the gastrointestinal tract). For example, a device that remains internal to a subject but transits the gastrointestinal tract over e.g., greater than or equal to 24 hours is not intended to be a device that is retained at a location internal to a subject for said greater than or equal to 24 hours, despite being internal to the subject. By way of example, an article that remains proximate the pylorus of the subject e.g., greater than or equal to 24 hours is intended to be considered an article that is retained at the location internal to the subject for said greater than or equal to 24 hours. Other residence time periods are also possible and are described in more detail below. Those of ordinary skill in the art would understand, based upon the teachings of this specification, that residence does not require a strict adherence to a geometrically defined relative to location internal to a subject such that the article may move (e.g., as a result of gastrointestinal forces/motion) while being retained at the location internal to the subject. By way of example, and without wishing to be bound by a literal interpretation of such, an article is said to be retained e.g., in the stomach of the subject as long as the structure remains in the stomach and does not exit the stomach (e.g., via the pylorus) during the desired residence time period. In some embodiments, the structures described herein comprise a component that undergoes a change (e.g., a mechanical change) such that the article exits the location internal to the subject (e.g., passes through the pylorus).

A “subject” refers to any animal such as a mammal (e.g., a human). Non-limiting examples of subjects include a human, a non-human primate, a cow, a horse, a pig, a sheep, a goat, a dog, a cat or a rodent such as a mouse, a rat, a hamster, a bird, a fish, or a guinea pig. Generally, the invention is directed toward use with humans. In some embodiments, a subject may demonstrate health benefits, e.g., upon administration of the articles described herein.

In some embodiments, the location internally of the subject is the colon, the duodenum, the ileum, the jejunum, the stomach, the small intestine, the large intestine, the rectum, the mouth, or the esophagus.

As described above and herein, in some embodiments, a pharmaceutical agent may be released during and/or after detection of radiation (or an undesired chemical agent) at the location internal to the subject.

In an exemplary set of embodiments, the article is administered such that the article enters the stomach of the subject and is retained in the stomach for a residence period (e.g., of greater than or equal to 24 hours). The article may be configured to sense and/or detect radiation and/or chemical toxin via one or more sensors. In some embodiments, the article may be configured to sense physiological conditions about the subject such as e.g., the presence of radiation, the presence of one or more chemical toxins, subject temperature (e.g., gastric internal temperature), local pH, local pressure, and/or other biophysical characteristics. For example, the article may comprise (and/or be in electronic communication with) one or more sensors configured to determine one or more physiological conditions about the subject. In some embodiments, the article comprises one or more sensors (e.g., a radiation sensor, a chemical toxin sensor, a biomolecular sensor, a gas sensor, a temperature sensor, a pressure sensor, a motion sensor, an accelerometer, a pH sensor, a biochemical sensor), a wireless identification microchip, and/or an imaging system (e.g., a camera). In some embodiments, the article is configured to generate and/or receive a signal from the one or more sensors and generate an electrical signal to one or more arms. In some embodiments, the signal triggers the article to change the one or more arms into a deployed configuration. In some embodiments, the signal triggers the article to reorient (e.g., via the one or more arms) such that a tissue interfacing component is positioned relative to a tissue located internal to a subject. In some embodiments, the signal triggers the article to delivery (e.g., via injection) the tissue interfacing component into the tissue located internal to the subject. In some embodiments, the tissue interfacing component delivers a therapeutic agent (e.g., to counteract the presence of a detected radiation and/or chemical toxin).

In some embodiments, the signal provides an orientation of the article to a microcontroller (e.g., for activation of the one or more arms). In some embodiments, the signal mediates the exit of the article from the stomach through the pylorus (e.g., after delivery of a therapeutic agent)

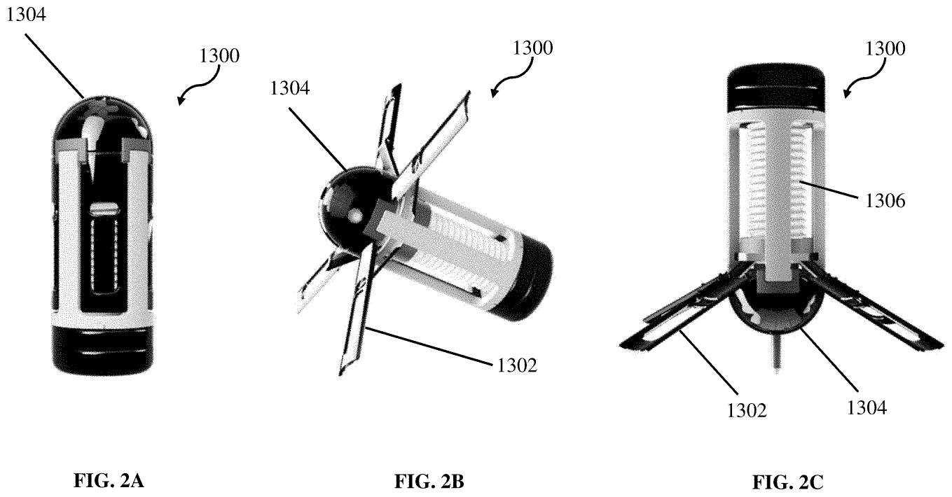

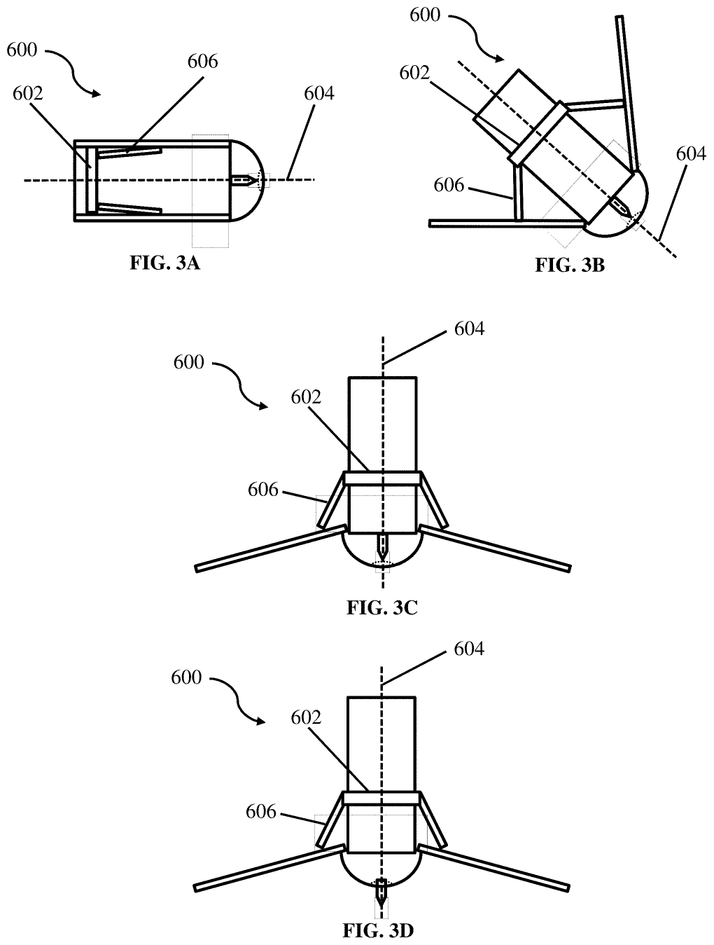

In some embodiments, a central core of the article comprises a tissue-engaging surface. The tissue-engaging surface may be associated with a drug release component, as described in greater detail below. According to certain embodiments, the injection mode of the article stabilizes the article such that the tissue-engaging surface contacts a tissue of a subject at a location internal to a subject. FIGS. 2A-2C present an exemplary embodiment of article 1300, which is shown in FIG. 2A in a retracted configuration. FIG. 2B shows article 1300 in an expanded configuration intended to facilitate long-term residence of the article within the gastrointestinal tract, and FIG. 2C presents article 1300 in an injection mode of the expanded configuration, wherein the arms 1302 of article 1300 are positioned to better stabilize the capsule, such that tissue-engaging surface 1304 can contact the tissue of the subject at the location internal to a subject.

In some embodiments, an article may be deployed in a body of a subject through ingestion. For example, an article may be enclosed within a capsule, such as a gelatin or other dissolvable capsule, in the retracted configuration. A subject may swallow the encapsulated article, introducing the article into the gastrointestinal tract. The capsule may be configured to dissolve after a predetermined time or upon reaching a predetermined environment, allowing the article to be deployed at a predetermined location within the body of the subject. However, in some embodiments, the article may be deployed in the body endoscopically, surgically, or in any other appropriate manner, as the disclosure is not limited in regards to the method of deploying the article to a desired location within a subject's body.

For example, as shown in FIG. 1A, a capsule 102, such as a dissolvable gelatin capsule, may at least partially surround the central core and the arms, retaining the arms in the retracted configuration prior to deployment. In this configuration, the L-shaped beams may be deformed such that elastic energy is stored in the structure and a biasing force is applied to the arms that biases the arms towards an expanded configuration as elaborated on below. While the elastic components and biasing elements are shown in the drawings as L-shaped beams and springs (respectively), it should be appreciated that any suitable geometries and/or structures may be used for the elastic components and the biasing components as the disclosure is not limited in this regard.

The arms may be rotatably coupled to the central core in any appropriate manner that permits the arms to pivot outward away from the central core to selectively reconfigure the article between a retracted configuration and an expanded configuration.

In some instances, the arms may rotate about an axis of rotation that is approximately perpendicular to a direction of a longitudinal axis of the central core. In some embodiments, such as the exemplary embodiments of FIGS. 1A-1C, beams 110 may be L-shaped beams that may couple two adjacent arms, such that each arm is perpendicular to its two adjacent arms. When the arms are brought into the retracted configuration, the L-shaped beams may be deformed relative to their undeformed neutral configuration. Thus, in a retracted configuration, the arms may be biased outwards away from the central core into the expanded configuration by the L-shaped beams or other elastic component. For example, a capsule 102 may retain the article in a retracted configuration (FIG. 1A). In such a configuration, beams 110 may be deformed, storing elastic energy. When the capsule is removed (e.g., when a gelatin capsule is dissolved in the esophagus), the stored elastic energy of the beams may cause the arms to unfold (FIG. 1B), causing the article to expand. In a fully expanded configuration (FIG. 1C), the arms may be substantially perpendicular to the long axis of the central core.

In the expanded configuration, one or more biasing components associated with each arm, i.e., biasing springs 108, may exert a torque on the arms 106 to urge the arms back towards the central core 104. A spring 108 may be coupled to the central core 104 on one side and to an arm 106 on an opposite side. The spring may be operatively coupled to the arm and/or the core through the use of an adhesive, an interference fit with a hole, simply being placed into contact with the arm with a portion of the arm disposed between the L-shaped beam or other elastic component and the spring, or any other suitable method of associating the biasing component with the corresponding arm.

Using such a configuration, when the biasing components (e.g., springs), are in a first state, the torque exerted on the arms by the biasing components (which may bias the arms towards the central core) may be less than the torque exerted on the arms by the elastic components 110 (which may bias the arms away from the core). As such, the article 100 may remain in an expanded configuration when the springs are in the first state. In contrast, when article 100 is in the second state, the plurality of arms 106 may become biased towards the central core 104. This may cause them to transition into the retracted configuration. Specifically, the torque exerted on the arms 106 by the biasing components 108 (which may bias the arms towards the central core) may be greater than the torque exerted on the arms 106 by the elastic components 110 (which may bias the arms away from the core), which may cause the arms to retract towards the central core. In some case, the transition between the first state and the second state of the biasing components may be actuated, as described in greater detail below.

In some embodiments, there may be competing forces exerted by the elastic beams (or other elastic component) and the biasing components. In some embodiments, the relative strength of the force applied by the biasing component may be increased in response to an external signal (e.g., a signal originating from outside the article). In other embodiments, the relative strength of the force applied by the biasing component may be increased in response to an external condition. For example, the force applied to the biasing component may be increased in response to an external force, or in response to an external chemical environment. Thus, the article may be biased toward the retracted configuration by increasing the force applied by the biasing components to be greater than the threshold temperature. This may correspondingly cause an overall force applied to the arms to be directed towards the core to bias the article into the retracted configuration. The biasing components may be actuated by any of a variety of methods recognized in the art. The article may be electromagnetically actuated. For example, the article may comprise an electromagnet that can be switched on remotely, in order to apply a biasing force to the arms. The article may be electro-mechanically actuated. In some embodiments, the biasing components are hydraulically actuated. In some embodiments, the biasing components are pneumatically activated. In various embodiments, the biasing components are chemically actuated. In some embodiments, the biasing components are piezoelectrically actuated. The biasing component may also be actuated thermally. For instance, in some embodiments, the biasing components may comprise biasing components that tend to deform in response to temperature changes.

Exemplary thermal actuators include shape-memory alloys and bimetallic strips. The biasing components may also be actuated by magnetostriction. In some embodiments, the biasing components are actuated by electroactive polymers. The biasing components are actuated by bladders, in some embodiments. According to certain embodiments, the biasing components are actuated by micro-electromechanical systems (MEMS). In some cases, all biasing components of an article are actuated identically. However, in other embodiments, combinations of distinct types of actuation may be used. For example, in some embodiments, some biasing components may be thermally activated, while other biasing components may be hydraulically actuated. The application is not so limited.

Biasing components may have any of a variety of appropriate forms. In some embodiments, a biasing component may comprise an actuated spring, a bimetallic strip, a U-shaped structure, an L-shaped structure, or any other suitably shaped structure extending between the core of an article and an associated arm, or another appropriately shaped component capable of applying a desired force. Further the various parameters of the component including material properties, dimensions, overall geometry, and other appropriate parameters may be selected to provide a desired restoring force upon actuation. Accordingly, it should be understood that a biasing component is not limited to only the specific structures described herein.

As an alternative to elastic components and biasing components, in some embodiments, the transition of the arms between the retracted and the expanded configuration is electro-mechanically actuated. For example, in some embodiments, an article comprises an electro-mechanical actuator configured to translate a rigid body in a direction parallel to a central axis of an article. For example, FIGS. 3A-3D present translation an exemplary embodiment of article 600 comprising rigid body 602 configured to be translated in a direction parallel to central axis 604 of article 600.

The rigid body may enclose at least part of a lateral cross-section of the central core of the article, in some embodiments. For example, the rigid body may totally enclose the lateral cross-section of the central core of the article. As another example, the rigid body may enclose an interior portion of a lateral cross-section of the central core of the article (e.g., the rigid body may enclose a lead screw of a motor, or may form a portion of a lead screw of a motor that encloses). The configuration of the rigid body to enclose a lateral cross-section of the central core may advantageously allow translation of the rigid body along the central axis of the article, in a direction parallel to the central axis of the article. As one example, in some embodiments, the central core comprises a cylindrical portion, and the rigid body is a ring or a cylinder surrounding the central core and centered on the central axis of the article. As another example, in some embodiments, the central core comprises a prismatic portion. For example, the prismatic portion may have a shape corresponding to a triangular prism, a rectangular prism, a hexagonal prism, or another prism having any other appropriate number of sides. In some embodiments the rigid body is a polygon corresponding to the prismatic portion of the central core. For example, in some embodiments the corresponding polygon may be a triangle, a rectangle, a hexagon, or another polygon corresponding to the cross-sectional dimensions of the prismatic portion.

The rigid body may be coupled to a proximal end of an arm-connector, in some embodiments. For example, in FIGS. 3A-3D, rigid body 602 is connected to arm connectors 606. In some embodiments, a distal end of the arm-connector is coupled to an arm of the plurality of arms of the article. As used herein, ‘coupling’ of two parts may be achieved either by direct connection between those parts, or via one or more intervening components. Exemplary intervening components that may be positioned between arm-connectors, arms, and/or rigid bodies, include hinges or sliders. The arm-connector may be rigid, such that translation of the rigid body in a direction parallel to the central axis of the article applies a force to the arm, causing it to rotate relative to the article. In some embodiments, the rigid body is connected to a plurality of arm connectors. The plurality of arm connectors may be connected at a plurality of distal ends to the plurality of arms of the article. Thus, motion of the rigid body may result in simultaneous rotation of the arms with respect to the article. For example, in FIGS. 3A-3D, arm connectors 606 are connected at distal ends to arms 610. Thus, motion of rigid body 602 may result in simultaneous rotation of arms 610. In some embodiments, at least one arm connector is connected to each arm of the plurality of arms of the article.

In some embodiments, the article is configured such that translation of the rigid body in a first direction parallel to the central axis of the article will cause the arms to transition to an expanded configuration. In some embodiments, the article is configurated such that translation of the rigid body in a second direction, opposite to the first direction, will cause the arms to transition to a closed configuration.

The rigid body may be translated by any of a variety of appropriate methods. In some embodiments, an electro-mechanical actuator may be used to translate the rigid body. For example, in some embodiments, a motor of the article may be used to translate the rigid body. The motor may be, for example, a DC geared motor. In some embodiments, the motor may comprise a lead screw. For example, the article of FIGS. 2A-2C comprises lead screw 1306. In some embodiments, the lead screw is configured to move the rigid body. In some embodiments, the lead screw is configured to actuate a drug release component as described below, e.g., by moving a locking mechanism, as described below. In some embodiments, a piezoelectric, electromagnetic, pneumatic, and/or hydraulic actuator may be used to translate the rigid body. The disclosure is not, in this way limited. It should also be understood that while annular rigid bodies corresponding to cross-sections of portions of the central core of the article are described herein, the rigid body is not limited to this shape. For example, the rigid body may have an X-shape, wherein the rigid body is translated via application of force to a center of the rigid body, while proximal ends of the arm-connectors are coupled to the X-shape away from the center of the X-shape. In some embodiments, the article comprises a plurality of rigid bodies. In certain embodiments, the rigid bodies of the plurality of rigid bodies of the article may be configured to be translated in a direction parallel to a central axis of the article. In some embodiments, rigid bodies of the plurality of rigid bodies are configured to change the configuration of one or more arms of the plurality of arms.

In some embodiments, an article described herein may orient itself during a change in configuration. For instance, the article descried herein may exhibit self-righting behavior during a transition from a retracted configuration to an expanded configuration. FIGS. 3A-3C demonstrate this behavior, in an exemplary embodiment. In FIG. 3A, exemplary article 600 has an initially horizontal orientation and is in the retracted configuration. In FIG. 3B, article 600 begins to transition from the retracted configuration to the expanded configuration, resulting in a change in orientation of central axis 604 of article 600. Finally, in FIG. 3C, article 600 is oriented such that central axis 604 is substantially aligned with a vertical axis. In some embodiments, an article in the expanded configuration is self-righting, as described in greater detail below.

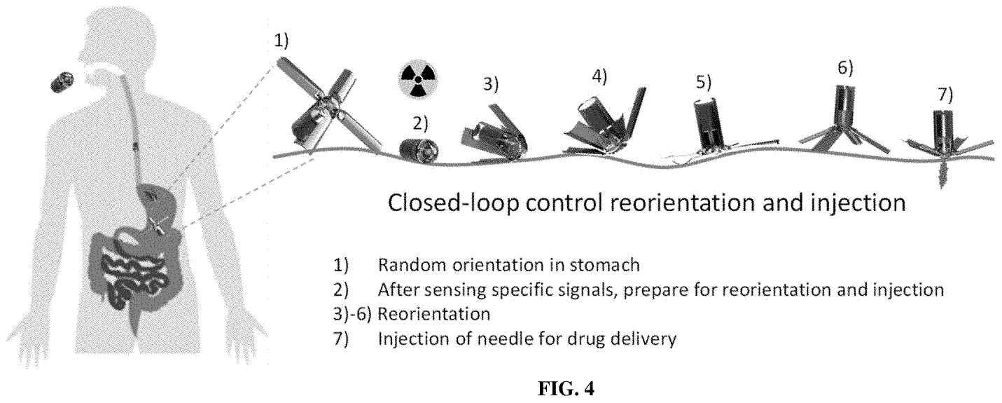

FIG. 4 presents an embodiment of a method of reorienting an article in greater detail than FIGS. 3A-3C. First, the article may be passed through the esophagus in the retracted configuration. Then, it can transition to an expanded configuration, e.g., for long-term gastric residence. The transition between the initial, retracted configuration and the expanded configuration may be triggered in response to a change in the external environment of the article (e.g., a change in pH, temperature, pressure, and/or humidity). Initially, the expanded article may have a random orientation in the stomach (step 1). After sensing specific signals (e.g., signals related to external environment, signals detected by one or more sensors of the article, or signals provided by an operator), the article may be transformed into a retracted configuration, in preparation for reorientation and injection (step 2). Next, the article may transition from the retracted configuration to the expanded configuration (steps 3-6). For example, the article may transition to an injection mode of the expanded configuration, as shown. During these steps, measurements from an accelerometer, a gyro and/or a magnetometer may allow the article to identify its orientation and the position of its arms. The accelerometer may be used to monitor the existence of gastric contraction. Data collected by the accelerometer may be used to determine a time suitable for use of the drug release component of the article. Finally, the drug release component (e.g., the needle, in this case) may be interfaced with tissue (e.g., via injection of the needle into the tissue). In some embodiments, the accelerometer may be used to detect release of the active pharmaceutical composition. Without wishing to be bound by theory, the accelerometer may be used to monitor release of the active pharmaceutical composition because, in some embodiments, release of the active pharmaceutical composition may produce a detectable vibration profile.

In some embodiments, the article may comprise an arm-locking component. The arm-locking component may be configured to hold one or more arms in a retracted configuration. For example, the arm-locking component may comprise a hydrogel configured to hold one or more arms in the retracted configuration. According to certain embodiments, the arm-locking component is chemically sensitive. For example, the arm-locking component may be configured to respond in the presence of a particular chemical (e.g., a toxic chemical, as described below). In some embodiments, the arm-locking component may be configured to respond to a change in pH. The arm-locking component may respond to the chemical and/or the change in pH by swelling, shrinking, and/or degrading. In some embodiments, the response of the arm-locking component to the chemical and/or the change in pH may result in the opening of the arms. For example, in some embodiments, the arm-locking mechanisms are configured to degrade in response to the chemical and/or the change in pH, releasing the arms into the expanded configuration.

While the use of an article in the esophagus of a subject is described above, it should be understood that the current disclosure is not limited to using articles only in the esophagus and/or gastrointestinal tract of a subject. For example, the disclosed articles may be used in any appropriate anatomical structure in the body where it may be desirable to have an article transition between an expanded configuration and a retracted configuration for delivery of an active pharmaceutical compound and/or sensing applications. Other appropriate types of anatomical structures where the disclosed medical articles may be used include, but are not limited to, a stomach, small intestine, large intestine, trachea, colon, ureters, urethra, and any tubular viscus of a subject.

It should be understood that the various components of an article may be made from any appropriate material compatible with the anatomical structures with which the article will interact during use and exhibiting appropriate properties for a desired application. In some embodiments, the arms and the central core may comprise any suitable material and may have appropriate dimensions to provide a desired rigidity during deployment and use. Appropriate materials may include, but are not limited to: polymeric materials such as poly(c-caprolactone) (PCL), thermoplastic polyurethanes (TPUs), poly(vinyl alcohol) (PVA), polylactic acid (PLA), poly(lactic-co-glycolic acid) (PLGA), silicone-based elastomers with high shore hardness, metals (e.g., titanium), and/or any other suitable material.

An elastic component may be made from any sufficiently elastic material and may have any appropriate construction to provide a desired biasing force to bias a article to an expanded configuration. Appropriate materials may include, but are not limited to: elastic and/or elastomeric polymers such as thermoplastic polyurethane, silicon-based elastomers, polydimethylsiloxane (PDMS), and other appropriate polymers; flexible metals such as stainless steel, titanium, alloys thereof, and other metals compatible with the anatomical structure with which the article may interact; and/or any other appropriate elastic material. Depending on the specific material and corresponding elasticity, the elastic components may take any number of different forms including, but not limited to, springs, beams, solid components, components with portions having reduced cross sections to form living hinges, and/or any other appropriate structure exhibiting a desired combination of flexibility and stiffness to provide the desired functionality.

In some applications, it may be desirable to be able to quickly reconfigure an article. Actuation time of the article may refer to the amount of time for the article to reconfigure from the expanded configuration to the retracted configuration action of actuated biasing components of the article. In some embodiments, the actuation time of the an article may be less than 10 minutes, 5 minutes, 1 minute, 10 seconds, and/or any other appropriate time period. Additionally, in some embodiments, the above-noted actuation times may be greater than 0.5 seconds, 1 second, and/or any other appropriate time period. Combinations of the foregoing are contemplated including, for example, actuation times that are between or equal to 0.5 seconds and 1 minute, 0.5 seconds and 10 minutes, and/or any other appropriate time period including time periods both greater and less than those noted above.

In some embodiments, an active pharmaceutical composition (e.g., comprising an active pharmaceutical agent such as a counter-radiation agent) is released from a drug release component of an article. The drug release component may be comprised by a central core of an article. For example, the drug release component may be associated with a tissue-engaging surface of the article. According to certain embodiments, the drug release component is a self-actuating component. However, in some embodiments, the drug release component is actuated by an electro-mechanical actuator (e.g., a motor) and/or a circuit as described elsewhere herein. Advantageously, the release components described herein may be useful as a general platform for delivery of a wide variety of active pharmaceutical compositions that are typically delivered via injection directly into tissue. For example, the drug release components may be configured to release a counter-radiation agent.

Self-actuating drug release components may include, for example, self-actuating tissue interfacing components such as self-actuating needles tissue-engaging component. In some embodiments, the article comprises a spring (e.g., a coil spring, wave springs, Belleville washers, a beam, a membrane, a material having particular mechanical recovery characteristics). The spring may, in some cases, be operably linked to the tissue interfacing component. Those of ordinary skill in the art would understand that the term spring is not intended to be limited to coil springs, but generally encompass any reversibly compressive material and/or component which, after releasing an applied compressive force on the material/component, the material/component substantially returns to an uncompressed length of the material/component under ambient conditions (e.g., within 80%, within 90%, within 95% of the length of the material/component prior to compression).

Externally-actuated drug release components may include, for example, tissue interfacing components such as needles. In some embodiments, the article comprises a spring as described above, which is configured to be released and/or compressed by a motor. As in the case of self-actuating drug release components, the spring may, in some cases, be operably linked to the tissue interfacing component. In some embodiments, externally-actuated drug release components comprise tissue interfacing components that are translated into tissue. For example, the tissue-interfacing component may inserted into tissue using a motor.

According to some embodiments, the drug release component is coupled to a drug reservoir component. For example, the drug release component may be connected to the drug reservoir component. It should be understood that as used herein, connected components may be directly connected (e.g., they may directly contact one another), or they may be indirectly connected (e.g., they may be separated via one or more intervening components). In some embodiments, the drug release component is directly connected to the drug reservoir component. In some embodiments, the drug reservoir component is removable from the drug release component. However, the drug reservoir component may be integrally formed with the drug release component, or may be a portion of the drug release component, in some embodiments. The disclosure is not so limited. The drug reservoir component may have any of a variety of suitable geometries, and may be configured to retain the active pharmaceutical composition. In some embodiments, the active pharmaceutical composition may be transmitted from the drug reservoir component to the drug release component, such that the drug reservoir component acts as an additional source of the active pharmaceutical composition. Although the drug reservoir component may be configured to retain the active pharmaceutical composition, this is not a requirement of all drug reservoir components. In some embodiments, the drug reservoir component does not comprise an active pharmaceutical composition. For example, the drug reservoir component may be empty. In some embodiments, the drug reservoir component is not configured to comprise an active pharmaceutical composition. For example, the drug reservoir component may simply be incorporated to provide an appropriate connection between the drug release component and the article. The disclosure is not so limited.

In some embodiments, the drug reservoir component is configured to retain the drug release component within the article until a time when the article is configured to deliver the active pharmaceutical composition. In a first configuration of the article, in some embodiments, the drug reservoir component is positioned against a compressed spring. Springs are described above, in greater detail. In some embodiments, the drug reservoir component is positioned against the compressed spring, such that the spring applies a first force to the drug reservoir component. For example, the spring may be positioned such that, in the absence of an opposing force, the spring would push the drug reservoir component and the drug release component away from the center of mass of the article.

In some embodiments, the article further comprises an anchoring component, designed to provide an opposing force to the compressed spring. The anchoring component may be configured to apply a second force to the drug reservoir component. The second force may at least partially oppose the first force. In the first configuration of the article, in some embodiments, the anchoring component may be configured such that a first portion of the anchoring component overlaps at least a portion of the drug reservoir component. For example, the snapping portion may be configured to at least partially overlap a perimeter of the drug reservoir component. In some embodiments, that the anchoring component provides a response force opposing the compressed spring. In some embodiments, the anchoring component can not, without reinforcement, provide enough force to oppose the motion of the drug reservoir component and the drug release component. As used herein, two surface portions are considered to overlap if a ray extending perpendicularly from the first surface portion would extend through the second surface portion.

According to certain embodiments, the article further comprises a locking mechanism. In some embodiments, the anchoring component is reinforced by the locking mechanism in the first configuration of the article. The locking mechanism may be configured to apply additional force opposing the expansion of the spring. For example, in some embodiments, the locking mechanism is configured to apply a third force to a second portion of the anchoring component, such that the third force at least partially opposes the first force. In some embodiments, in the first configuration, the first force, the second force, and the third force hold the drug reservoir component in static equilibrium with respect to the article. The second portion of the anchoring component may be located on an opposite side of the anchoring component from the first side of the anchoring component. In some embodiments, the article can be transitioned from a first configuration to a second configuration by removing the locking mechanism from contact with the snapping portion. According to certain embodiments, the second force is insufficient to oppose the first force. In some embodiments, removal of the locking mechanism from contact with the anchoring component results in expansion of the spring. In some embodiments, removal of the locking mechanism from contact with the anchoring component results in release of the drug delivery article from the anchoring component. In some embodiments, an active pharmaceutical composition may be administered to a subject by transitioning the article first configuration to the second configuration. In some embodiments, the drug release component may contact tissue in the second configuration.

The locking mechanism may, in some embodiments, be mechanically coupled to an electro-mechanical actuator of the article. According to certain embodiments, the electro-mechanical actuator may comprise a motor. In some embodiments, the electro-mechanical actuator may be actuated by a circuit. The circuit may be configured to actuate the drug release component by moving the locking mechanism. For example, in some embodiments, the locking mechanism may be moved away from the anchoring component, such that it ceases to contact the anchoring component. According to certain embodiments, moving the locking mechanism away from the anchoring component may allow the drug reservoir component to move past the anchoring component (e.g., in response to the force applied by the spring).

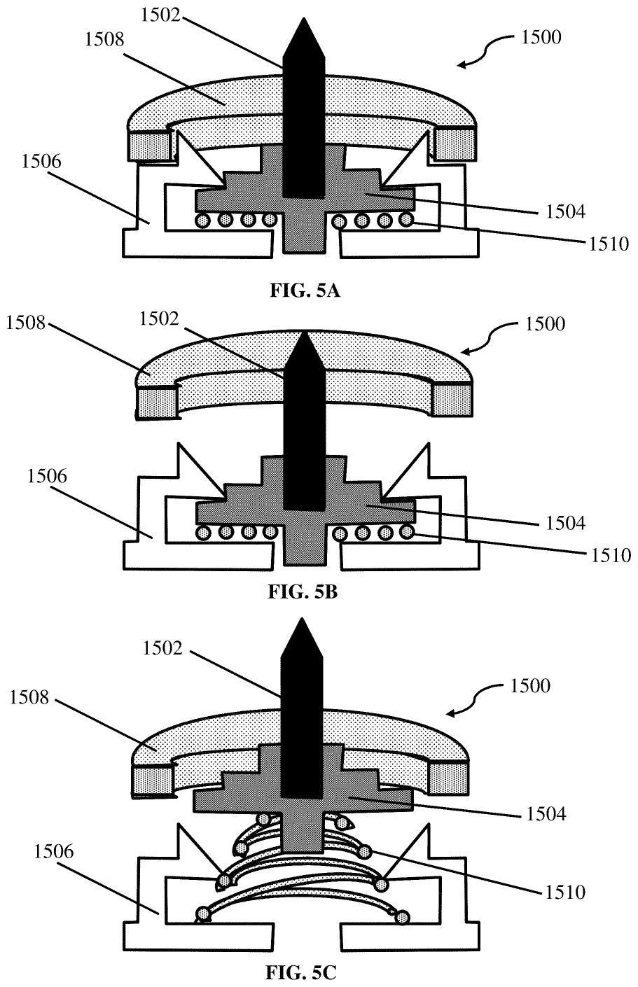

FIGS. 5A-5C illustrate the drug release component 1502 of exemplary article 1500, which is connected to drug reservoir component 1504. In FIG. 5A, the article is in the first configuration, and drug reservoir component 1504 is held in place by anchoring component 1506, which is reinforced in turn by locking mechanism 1508. In FIG. 5A, spring 1510 is configured to push drug reservoir component 1504 upon the release of spring 1510. However, in FIG. 5A, spring 1510 is retained in a compressed state by the anchoring component and the locking mechanism.

FIG. 5B, meanwhile, presents the transition of article 1500 from the first configuration of FIG. 5A to the second configuration, via actuation of locking mechanism 1508 by an electro-mechanical actuator (not shown). The electro-mechanical actuator (e.g., the motor) may be actuated by a circuit (not shown), as described in greater detail elsewhere herein. In some embodiments, the actuation of the locking mechanism may be performed in response to a signal (e.g., a signal sent from an external operator, or a signal resulting from an external condition, such as an increase in radiation, detected by a sensor). Once locking mechanism 1508 is removed from anchoring component 1506, anchoring component 1506 may be unable to provide sufficient force to oppose the force provided by spring 1510. As a result, drug reservoir component 1504 may be released from anchoring component 1506 when the article is in the second configuration by the expansion of spring 1510, as illustrated in FIG. 5C.

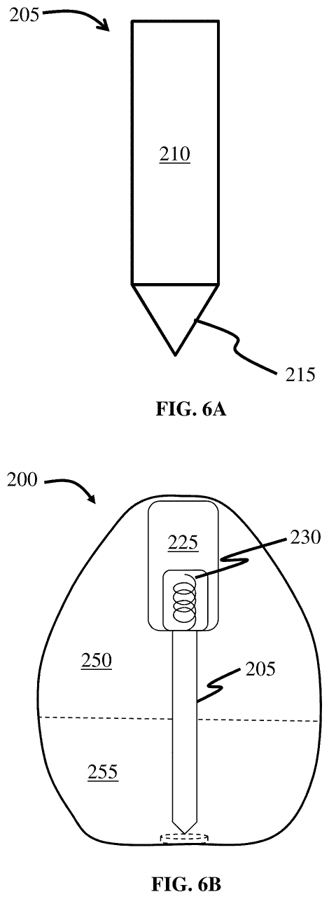

In some embodiments, a tissue interfacing component of an article comprises a base portion and a tip. For example, as illustrated in FIG. 6A, tissue interfacing component 205 comprises base portion 210 and tip 215. In some embodiments, the base portion comprises a mucoadhesive polymer. In some embodiments, the base portion and/or the tip comprises an active pharmaceutical composition (e.g., comprising an active pharmaceutical agent such as a counter-radiation agent) and a second material (if present), such that the active pharmaceutical composition is present in the tissue interfacing component in an amount of greater than or equal to 10 wt % versus the total weight of the tissue interfacing component. In certain embodiments, the active pharmaceutical composition is present in the tissue interfacing component in an amount of greater than or equal to 10 wt %, greater than or equal to 20 wt %, greater than or equal to 30 wt %, greater than or equal to 40 wt %, greater than or equal to 50 wt %, greater than or equal to 60 wt %, greater than or equal to 70 wt %, greater than or equal to 80 wt %, greater than or equal to 90 wt %, greater than or equal to 95 wt %, greater than or equal to 98 wt %, or greater than or equal to 99.1 wt % versus the total weight of the tissue interfacing component. In some embodiments, the active pharmaceutical composition is present in the tissue interfacing component in an amount of less than or equal to 100 wt %, less than or equal to 99 wt %, less than or equal to 98 wt %, less than or equal to 95 wt %, less than or equal to 90 wt %, less than or equal to 80 wt %, less than or equal to 70 wt %, less than or equal to 60 wt %, less than or equal to 50 wt %, less than or equal to 40 wt %, less than or equal to 30 wt %, or less than or equal to 20 wt % versus the total weight of the tissue interfacing component. Combinations of the above-referenced ranges are also possible (e.g., greater than or equal to 10 wt % and less than or equal to 100 wt %, greater than or equal to 80 wt % and less than or equal to 100 wt %). Other ranges are also possible. In an exemplary set of embodiments, the active pharmaceutical composition is present in the tissue interfacing component in an amount greater than or equal to 20 wt % and less than or equal to 80 wt % versus the total weight of the tissue interfacing component.

In some embodiments, the spring may instead be mechanically, electro-mechanically, and/or electrically actuated. In some cases, mechanical, electro-mechanical, and/or electrical actuation of the spring may be performed in response to an external signal (e.g., delivered from a remote operator to the article). In some embodiments, mechanical, electro-mechanical, and/or electrical actuation of the spring may be performed in response to a change in external conditions (e.g., a change in ambient radiation conditions).

In some embodiments, the article maintains at least a portion of the spring under at least 5%, at least 10%, at least 15%, at least 20%, at least 25%, at least 30%, at least 40%, at least 50%, at least 60%, at least 70%, or at least 80% compressive strain. In certain embodiments, the article maintains at least a portion of the spring under less than or equal to 90%, less than or equal to 80%, less than or equal to 70%, less than or equal to 60%, less than or equal to 50%, less than or equal to 40%, less than or equal to 30%, less than or equal to 25%, less than or equal to 20%, less than or equal to 15%, or less than or equal to 10% compressive strain.

In certain embodiments, the spring recovers (e.g., within less than 10 minutes, less than 5 minutes, less than 1 minute, less than 30 seconds, less than 10 seconds, less than 5 seconds, less than 1 second) to a length of greater than or equal to 80%, greater than or equal to 85%, greater than or equal to 90%, greater than or equal to 95%, greater than or equal to 98%, or greater than or equal to 99% of the length of the spring (e.g., an uncompressed spring length) prior to applying and/or in the absence of the compressive strain (e.g., by the locking mechanism and/or the anchoring component). In some embodiments, the spring recovers to a length of less than or equal to 100%, less than or equal to 99%, less than or equal to 98%, less than or equal to 95%, less than or equal to 90%, or less than or equal to 85% of the length of the spring prior to applying and/or in the absence of the compressive strain.

Advantageously, the use of springs as described herein may allow, for example, the release of a tissue interfacing component (e.g., a needle) associated with (e.g., operably linked with) the spring such that the tissue interfacing component contacts and/or penetrates tissue proximate the article. In an illustrative example, in some embodiments, a needle associated with the spring is administered to a subject such that, upon release of a locking mechanism from a snapping mechanism, the spring recovers and the needle is pushed into tissue proximate the article such that the needle penetrates the tissue (e.g., a GI mucosal layer).

In some such embodiments, an active pharmaceutical ingredient is delivered into the tissue by the tissue interfacing components. For example, in some embodiments, the article comprises an active pharmaceutical ingredient such that, upon release of the spring at a location internal of a subject, the active pharmaceutical ingredient is released (e.g., into tissue proximate the location internal of the subject). Referring again to FIG. 6B, in some embodiments, central core 200 comprises tissue interfacing component 205 associated with spring 230. Tissue interfacing components (e.g., needles) are described in more detail, herein.

In some embodiments, a tissue interfacing component comprises an active pharmaceutical ingredient. In some embodiments, the tissue interfacing component comprises greater than or equal to 0 wt %, greater than or equal to 75 wt %, greater than or equal to 80 wt %, greater than or equal to 85 wt %, greater than or equal to 90 wt %, greater than or equal to 95 wt %, or more of an active pharmaceutical agent (e.g., a counter-radiation agent). In some embodiments, the tissue interfacing component comprises less than or equal to 99 wt %, less than or equal to 95 wt %, less than or equal to 90 wt %, less than or equal to 85 wt %, less than or equal to 80 wt %, less than or equal to 75 wt %, or less of an active pharmaceutical agent. Combinations of these ranges are possible. For example, in some embodiments, the tissue interfacing component comprises greater than or equal to 70 wt % and less than or equal to 99 wt % of an active pharmaceutical agent.

In some embodiments, the article (e.g., the self-righting article) may be configured to anchor to a location internal to a subject (e.g., a tissue at a location internal to a subject). As described above, in some embodiments, the self-righting article comprises one or more tissue interfacing components comprising one or more tissue-engaging components. In another exemplary embodiment, the article may comprise a spring and at least one tissue-engaging component operably linked to the spring. Springs are described in more detail, below. Other embodiments are also possible comprising at least one tissue-engaging component associated with a self-righting article and/or a self-actuating component.

In some embodiments, the tissue-engaging component (and/or the article comprising the tissue-engaging component) is configured to be retained at a location internal to a subject. For example, in some embodiments, the tissue-engaging component engages with a surface (e.g., a surface of a tissue) at the location internal to the subject such that it is retained at that location.

Advantageously, the systems comprising one or more tissue-engaging components described herein may be inserted into a surface of tissue at a location internal to a subject, and may maintain contact with the tissue under relatively high applied forces and/or relatively high change in orientation (e.g., by compressive forces exerted by the gastrointestinal tract and/or under high flow rates within the gastrointestinal tract). In some embodiments, the systems described herein do not substantially block orifices within the gastrointestinal tract (e.g., in the pylorus) e.g., restricting flow and enabling longer contact times. Natural replenishment of the walls of the gastrointestinal tract may permit desirable detachment and/or expulsion of the systems and articles described herein, without the need for surgical and/or endoscopic retrieval.

For example, in some embodiments, the tissue-engaging component is inserted into a surface of a tissue at a location internal to a subject and maintains contact with the tissue (e.g., the article remains anchored) under a change of orientation of the article of greater than or equal to 1 degree, greater than or equal to 2 degrees, greater than or equal to 5 degrees, greater than or equal to 10 degrees, greater than or equal to 15 degrees, greater than or equal to 20 degrees, greater than or equal to 25 degrees, or greater than or equal to 30 degrees. In certain embodiments, the article may remain anchored under a change of orientation of the article of less than or equal to 45 degrees, less than or equal to 30 degrees, less than or equal to 25 degrees, less than or equal to 20 degrees, less than or equal to 15 degrees, less than or equal to 10 degrees, less than or equal to 5 degrees, or less than or equal to 2 degrees. Combinations of the above-referenced ranges are also possible (e.g., greater than or equal to 1 degree and less than or equal to 45 degrees, greater than or equal to 2 degrees and less than or equal to 30 degrees). Other ranges are also possible.

In certain embodiments, the article (e.g., comprising the tissue-engaging component) is configured to be retained at the location internal to the subject under a normal retention force of greater than or equal to 0.002 N, greater than or equal to 0.004 N, greater than or equal to 0.006 N, greater than or equal to 0.008 N, greater than or equal to 0.01 N, greater than or equal to 0.012 N, greater than or equal to 0.014 N, greater than or equal to 0.016 N, greater than or equal to 0.018 N, greater than or equal to 0.02 N, greater than or equal to 0.025 N, greater than or equal to 0.03 N, greater than or equal to 0.04 N, greater than or equal to 0.05 N, greater than or equal to 0.1 N, greater than or equal to 0.15 N, greater than or equal to 0.2 N, greater than or equal to 0.25 N, greater than or equal to 0.3 N, greater than or equal to 0.35 N, greater than or equal to 0.4 N, greater than or equal to 0.5 N, greater than or equal to 0.6 N, greater than or equal to 0.7 N, greater than or equal to 0.8 N, or greater than or equal to 0.9 N of normally applied force per tissue-engaging component. In some embodiments, the article has a normal retention force of less than or equal to 1 N, less than or equal to 0.9 N, less than or equal to 0.8 N, less than or equal to 0.7 N, less than or equal to 0.6 N, less than or equal to 0.5 N, less than or equal to 0.4 N, less than or equal to 0.35 N, less than or equal to 0.3 N, less than or equal to 0.25 N, less than or equal to 0.2 N, less than or equal to 0.15 N, less than or equal to 0.1 N, less than or equal to 0.05 N, less than or equal to 0.04 N, less than or equal to 0.03 N, less than or equal to 0.025 N, less than or equal to 0.02 N, less than or equal to 0.018 N, less than or equal to 0.016 N, less than or equal to 0.014 N, less than or equal to 0.012 N, less than or equal to 0.01 N, less than or equal to 0.008 N, less than or equal to 0.006, or less than or equal to 0.004 N of normally applied force per tissue-engaging component. Combinations of the above referenced ranges are also possible (e.g., greater than or equal to 0.002 N and less than or equal to 1 N, greater than or equal to 0.02 N and less than or equal to 0.08 N, greater than or equal to 0.1 N and less than or equal to 1 N). Other ranges are also possible. The normal retention force as described herein may be determined by inserting the tissue-engaging component of the article into a surface of tissue (e.g., ex vivo swine stomach) to a penetration depth of at least 0.9 mm and then pulling the article, in a direction orthogonal to the surface of the tissue until the article dislodges from the tissue. The maximum force before dislodging the article is the normal retention force.

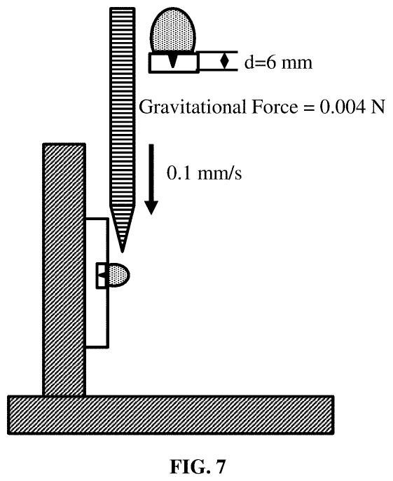

In some embodiments, the article (e.g., comprising the tissue-engaging component) is configured to be retained at the location internal to the subject under an orthogonal retention force of greater than or equal to 0.002 N, greater than or equal to 0.004 N, greater than or equal to 0.006 N, greater than or equal to 0.008 N, greater than or equal to 0.01 N, greater than or equal to 0.012 N, greater than or equal to 0.014 N, greater than or equal to 0.016 N, greater than or equal to 0.018 N, greater than or equal to 0.02 N, greater than or equal to 0.025 N, greater than or equal to 0.03 N, greater than or equal to 0.04 N, greater than or equal to 0.05 N, greater than or equal to 0.1 N, greater than or equal to 0.15 N, greater than or equal to 0.2 N, greater than or equal to 0.25 N, greater than or equal to 0.3 N, greater than or equal to 0.35 N, greater than or equal to 0.4 N, greater than or equal to 0.5 N, greater than or equal to 0.6 N, greater than or equal to 0.7 N, greater than or equal to 0.8 N, or greater than or equal to 0.9 N of normally applied force per tissue-engaging component. In some embodiments, the article has an orthogonal retention force of less than or equal to 1 N, less than or equal to 0.9 N, less than or equal to 0.8 N, less than or equal to 0.7 N, less than or equal to 0.6 N, less than or equal to 0.5 N, less than or equal to 0.4 N, less than or equal to 0.35 N, less than or equal to 0.3 N, less than or equal to 0.25 N, less than or equal to 0.2 N, less than or equal to 0.15 N, less than or equal to 0.1 N, less than or equal to 0.05 N, less than or equal to 0.04 N, less than or equal to 0.03 N, less than or equal to 0.025 N, less than or equal to 0.02 N, less than or equal to 0.018 N, less than or equal to 0.016 N, less than or equal to 0.014 N, less than or equal to 0.012 N, less than or equal to 0.01 N, less than or equal to 0.008 N, less than or equal to 0.006, or less than or equal to 0.004 N of normally applied force per tissue-engaging component. Combinations of the above referenced ranges are also possible (e.g., greater than or equal to 0.002 N and less than or equal to 1 N, greater than or equal to 0.02 N and less than or equal to 0.08 N, greater than or equal to 0.1 N and less than or equal to 1 N). Other ranges are also possible. The orthogonal retention force as described herein may be determined by inserting the tissue-engaging component of the article into a surface of tissue (e.g., ex vivo swine stomach) to a penetration depth of at least 0.9 mm and then applying a force to the article (see e.g., FIG. 7), in a direction parallel to the surface of the tissue, until the article dislodges from the tissue. The maximum force before dislodging the article is the orthogonal retention force.

In some embodiments, the article is configured to remain anchored to the surface of the tissue located internal to the subject under less than or equal to 30 degrees change in orientation and less than or equal to 1 N of applied (e.g., normal, orthogonal) force.

In some embodiments, the article comprises two or more tissue-engaging components. In some cases, the article may comprise a single article comprising two or more tissue-engaging components. In certain embodiments, the force required to dislodge the article (e.g., the normal retention force, the orthogonal retention force) may be increased by increasing the number of tissue-engaging components associated with the article. Without wishing to be bound by theory, the spacing between tissue-engaging components may be related to the retention force (e.g., the normal retention force, the orthogonal retention force) of the article.

The tissue-engaging component may have any suitable dimension and/or shape. For example, in some embodiments, the largest dimension (e.g., the length) of the tissue interfacing component comprising the tissue-engaging component may be less than or equal to 1 cm, less than or equal to 0.8 cm, less than or equal to 0.6 cm, less than or equal to 0.5 cm, or less than or equal to 0.4 cm. In certain embodiments, the largest dimension (e.g., the length) of the tissue interfacing component comprising the tissue-engaging component may be greater than or equal to 0.2 cm, greater than or equal to 0.4 cm, greater than or equal to 0.5 cm, greater than or equal to 0.6 cm, or greater than or equal to 0.8 cm. Combinations of the above-referenced ranges are also possible (e.g., greater than or equal to 0.2 cm and less than or equal to 1 cm). Other ranges are also possible.

In some cases, the tissue-engaging component may be configured to have an optimal penetration depth (e.g., the depth at which the tissue-engaging component is disposed beneath the surface of a tissue located internal to a subject). In some embodiments, the tissue-engaging component has a penetration depth of greater than or equal to 0.5 mm, greater than or equal to 0.6 mm, greater than or equal to 0.7 mm, greater than or equal to 0.8 mm, greater than or equal to 0.9 mm, greater than or equal to 1 mm, greater than or equal to 1.2 mm, greater than or equal to 1.4 mm, greater than or equal to 1.5 mm, greater than or equal to 1.7 mm, greater than or equal to 1.9 mm, greater than or equal to 2 mm, greater than or equal to 2.2 mm, greater than or equal to 2.4 mm, greater than or equal to 2.5 mm, greater than or equal to 3 mm, greater than or equal to 3.5 mm, greater than or equal to 4 mm, greater than or equal to 4.5 mm, or greater than or equal to 5 mm. In certain embodiments, the tissue-engaging component has a penetration depth of less than or equal to 6 mm, less than or equal to 5 mm, less than or equal to 4.5 mm, less than or equal to 4 mm, less than or equal to 3.5 mm, less than or equal to 3 mm, less than or equal to 2.5 mm, less than or equal to 2.4 mm, less than or equal to 2.2 mm, less than or equal to 2 mm, less than or equal to 1.9 mm, less than or equal to 1.7 mm, less than or equal to 1.5 mm, less than or equal to 1.4 mm, less than or equal to 1.2 mm, less than or equal to 1 mm, less than or equal to 0.9 mm, less than or equal to 0.8 mm, less than or equal to 0.7 mm, or less than or equal to 0.6 mm. Combinations of the above-referenced ranges are also possible (e.g., greater than or equal to 0.5 mm and less than or equal to 6 mm, greater than or equal to 0.9 mm and less than or equal to 2.5 mm). Other ranges are also possible.

Advantageously, the articles comprising an tissue-engaging component described herein may be retained for a relatively long period of time under physiological conditions and fluid flows (e.g., exposed to a fluid flowing at approximately 0.1 m/s). For example, in some embodiments, the article comprising an tissue-engaging component is retained at a surface of tissue located internal to a subject for greater than or equal to 1 hour, greater than or equal to 2 hours, greater than or equal to 4 hours, greater than or equal to 8 hours, greater than or equal to 12 hours, greater than or equal to 24 hours, greater than or equal to 2 days, greater than or equal to 3 days, greater than or equal to 5 days, greater than or equal to 7 days, or greater than or equal to 10 days. In certain embodiments, the article is retained for less than or equal to 14 days, less than or equal to 10 days, less than or equal to 7 days, less than or equal to 5 days, less than or equal to 3 days, less than or equal to 2 days, less than or equal to 24 hours, less than or equal to 12 hours, less than or equal to 8 hours, less than or equal to 4 hours, or less than or equal to 2 hours. Combinations of the above referenced ranges are also possible (e.g., greater than or equal to 1 hour and less than or equal to 14 days). Other ranges are also possible.

The tissue-engaging components described herein may comprise any suitable material. In some embodiments, the tissue-engaging component material is non-degradable.

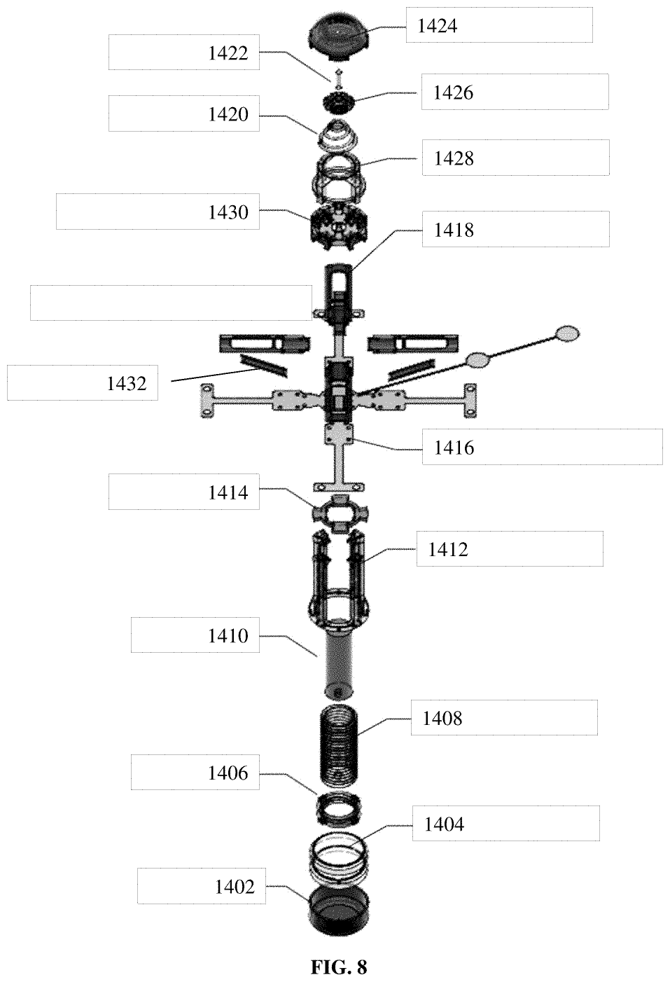

FIG. 8 presents an exploded-perspective schematic illustration of the exemplary article presented in FIGS. 2A-2C. As shown, the article 1300 comprises battery cap 1402, battery 1404, nut 1406, lead screw 1408, motor 1410, main body frame 1412, rigid component 1414, circuit 1416, arms 1418, spring 1420, drug release component 1422, cap 1424, drug reservoir component 1426, locking mechanism 1428, anchoring component 1430, and arm-connectors 1432.

In some embodiments, the article is self-righting. The self-righting article generally has a geometric center. In certain embodiments, the density, mass, and/or volume of the first portion and/or the second portion is selected such that the self-righting article exhibits self-righting behavior. According to certain embodiments, an article exhibits self-righting behavior via transitioning from a retracted configuration to an expanded configuration. For example, in some embodiments, an article in the retracted configuration may support itself using one or more of its arms, as it transitions from the retracted configuration to the expanded configuration. The transition from the retracted configuration to the expanded configuration may result in rotation of the central axis of the article, self-righting the article. The process is exemplified in FIGS. 3A-3D, which show a transition of the article from a fully retracted configuration (FIG. 3A) to a fully expanded configuration (FIG. 3C). In some embodiments, following self-righting, a drug release component can be extended from a tissue-interfacing surface, such that it can be exposed to tissue, as shown in FIG. 3D.

It should be understood that in some embodiments, the arms of the article may operate independently. Therefore, in some embodiments, the transition from the retracted configuration to the expanded configuration occurs as a result of a transition of some (but not all) arms to an expanded state. Thus, in some embodiments, a subset of the arms of the plurality of arms may be used to self-right the article.

In some embodiments the self-righting article is characterized as having a particular self-righting time from 90 degrees in a particular fluid. The self-righting time may be determined by placing the self-righting article in the particular fluid at 90 degrees, and allowing the self-righting article to return to a particular orientation otherwise maintained by the self-righting article in the absence of the fluid (e.g., an orientation corresponding to a stable point of equilibrium (or orientation) of the article).

In certain embodiments, the fluid is oil. In some such embodiments, the self-righting article has a self-righting time from 90 degrees in oil of less than or equal to 0.15 seconds, less than or equal to 0.1 seconds, less than or equal to 0.05 seconds, or less than or equal to 0.02 seconds. In certain embodiments, the self-righting article has a self-righting time from 90 degrees in oil of greater than or equal to 0.01 seconds, greater than or equal to 0.02 seconds, greater than or equal to 0.05 seconds, greater than or equal to 0.1 seconds, or greater than or equal to 0.12 seconds. Combinations of the above referenced ranges are also possible (e.g., less than or equal to 0.15 seconds and greater than or equal to 0.01 seconds). Other ranges are also possible. Self-righting time in oil is determined with the system/article fully submerged.

In some embodiments, the fluid is gastric fluid. In some such embodiments the self-righting article has a self-righting time from 90 degrees in gastric fluid of less than or equal to 0.06 seconds, less than or equal to 0.05 seconds, less than or equal to 0.04 seconds, less than or equal to 0.03 seconds, or less than or equal to 0.02 seconds. In certain embodiments, the self-righting article has a self-righting time from 90 degrees in gastric fluid of greater than or equal to 0.005 seconds greater than or equal to 0.01 seconds, greater than or equal to 0.02 seconds, greater than or equal to 0.03 seconds, greater than or equal to 0.04 seconds, or greater than or equal to 0.05 seconds. Combinations of the above referenced ranges are also possible (e.g., less than or equal to 0.06 seconds and greater than or equal to 0.005 seconds). Other ranges are also possible. Self-righting time in gastric fluid is determined with the system/article fully submerged.

In certain embodiments, the fluid is mucus. In some such embodiments the self-righting article has a self-righting time from 90 degrees in mucus of less than or equal to 0.05 seconds, less than or equal to 0.04 seconds, less than or equal to 0.03 seconds, or less than or equal to 0.02 seconds. In certain embodiments, the self-righting article has a self-righting time from 90 degrees in mucus of greater than or equal to 0.005 seconds greater than or equal to 0.01 seconds, greater than or equal to 0.02 seconds, greater than or equal to 0.03 seconds, greater than or equal to 0.04 seconds, or greater than or equal to 0.045 seconds. Combinations of the above referenced ranges are also possible (e.g., less than or equal to 0.05 seconds and greater than or equal to 0.005 seconds). Other ranges are also possible. Self-righting time in mucus is determined with the system/article fully submerged.

In some embodiments, the fluid is water. In some such embodiments the self-righting article has a self-righting time from 90 degrees in water of less than or equal to 0.05 seconds, less than or equal to 0.04 seconds, less than or equal to 0.03 seconds, or less than or equal to 0.02 seconds. In certain embodiments, the self-righting article has a self-righting time from 90 degrees in water of greater than or equal to 0.005 seconds greater than or equal to 0.01 seconds, greater than or equal to 0.02 seconds, greater than or equal to 0.03 seconds, greater than or equal to 0.04 seconds, or greater than or equal to 0.045 seconds. Combinations of the above referenced ranges are also possible (e.g., less than or equal to 0.05 seconds and greater than or equal to 0.005 seconds). Other ranges are also possible. Self-righting time in water is determined with the system/article fully submerged.

In some embodiments, the article comprises one or more vents (e.g., to permit the flow of air and/or fluid through the self-righting article). In some embodiments, the self-righting article comprises one or more (e.g., two or more, three or more, four or more) vents associated with at least a portion (e.g., the first portion, the second portion) of the article. In some such embodiments, the vent permits a fluid (e.g., gastric fluid) to enter at least a portion of the article such that e.g., the self-actuating component and/or the spring are exposed to the fluid (e.g., such that the self-actuating component and/or the spring actuate). In some embodiments, the article has a particular largest cross-sectional dimension. In some embodiments, the largest cross-sectional dimension of the article is less than or equal to 30 cm, less than or equal to 20 cm, less than equal to 10 cm, less than or equal to 5 cm, less than or equal to 2 cm, less than or equal to 1.1 cm, less than or equal to 1 cm, less than or equal to 0.8 cm, or less. In certain embodiments, the largest cross-sectional dimension of the article is greater than or equal to 0.1 cm, greater than or equal to 0.2 cm, greater than or equal to 0.4 cm, greater than or equal to 0.6 cm, greater than or equal to 0.8 cm, greater than or equal to 1 cm, greater than or equal to 1.1 cm, greater than or equal to 2 cm, greater than or equal to 2 cm, greater than or equal to 5 cm, greater than or equal to 10 cm, greater than or equal to 20 cm, or greater. Combinations of the above referenced ranges are also possible. For example, in some embodiments, the largest cross-sectional dimension of the article is greater than or equal to 0.1 cm and less than or equal to 30 cm. Other ranges are also possible.

In some embodiments, the article is administered (e.g., orally) to a subject. In some such embodiments, the article may comprise one or more active pharmaceutical ingredients. In certain embodiments, the active pharmaceutical ingredient is released at a location internal of the subject (e.g., within the G.I. tract).

In certain embodiments, one or more sensors are associated with the article, as described in greater detail below.

In some cases, one or more of the first portion and/or second portion may be magnetic.

The article may comprise a circuit, in some embodiments. For example, the article may comprise a circuit configured to control an electro-mechanical actuator, such as a motor. In some embodiments, the circuit is a printed circuit. For example, the circuit may be printed onto a flexible substrate. In some embodiments, the circuit has a Kirigami design, meaning that it may be prepared from an initially flat configuration via operations of cutting and/or folding. According to certain embodiments, the Kirigami-design may be expanded for gastric residence, self-orientation, radiation detection, or chemical detection

In some embodiments, the circuit comprises a processor. The processor may be a microcontroller unit (MCU). Processors are described in greater detail elsewhere herein. According to certain embodiments, the processor may be configured to control and/or interact with circuit portions. For example, in some embodiments, an actuator in electrical communication with a microcontroller may be opened or closed based upon a signal received from the microcontroller. Advantageously, for example, the actuator being electrically controllable by a microcontroller may avoid false-positive detections and/or undesired openings or closings.

In some embodiments, the circuit comprises a signal processing portion. The signal processing portion may be configured to receive an external signal. The external signal may be sent by a remote operator. For example, the external signal may be a radio frequency (RF) signal. In some embodiments, the external signal may be used to trigger a change in configuration of an article. For example, the external signal may be used to trigger a transition between a retracted configuration and an expanded configuration. Any appropriate triggering mechanism may be used. For example, the circuit may comprise a switch that may be actuated by the external signal, triggering action by an electro-mechanical actuator. In some embodiments, actuation of a switch by an external signal may result in a change in state of the article. In some embodiments, the signal processing portion comprises a transmitter. The transmitter may be configured to transmit signals (e.g., RF signals) to an external controller unit. In some embodiments, the transmitter may be configured to transmit signals to an operator (e.g., via an external controller unit). In some embodiments, an operator may operate an article based, at least in part, on information transmitted by the article. Information transmitted by the article may include radiation information, temperature information, pH information, chemical information, information regarding a state of the article, information regarding an orientation of the article, and/or any other appropriate form of information that may be determined by the article.

In some embodiments, the comprises a motor control portion. The motor control portion may be configured to actuate an electro-mechanical actuator (e.g., a motor). In some embodiments, an electro-mechanical actuator may be controlled by an external operator using the motor control portion of the circuit. For example, the motor control portion may be configured to actuate an electro-mechanical actuator used to transition an article between a retracted configuration and an expanded configuration. The motor control portion may be configured to actuate an electro-mechanical actuator that allows a drug release component to interface with tissue, in some embodiments. According to certain embodiments, the motor control portion may be configured to respond to a signal produced by a sensor. For example, in some embodiments, the motor control portion is configured to actuate a drug release component, at least in part in response to a signal received by a sensor.

In some embodiments, the motor control portion is configured to actuate more than one electro-mechanical actuator. For example, the motor control portion may be configured to actuate a first electro-mechanical actuator (e.g., a first motor) and a second electro-mechanical actuator (e.g., a second motor), in some embodiments. The motor control portion is configured to actuate a first electro-mechanical actuator that can change a configuration of the article between a retracted configuration and an expanded configuration, in some embodiments. The motor control portion is configured to actuate a second electro-mechanical actuator that can actuate a drug release component, in some embodiments. In some embodiments, the motor control portion is configured to actuate the first electro-mechanical actuator in response to an external signal and the second electro-mechanical actuator in response to a signal produced from a sensor of the article. For example, in some embodiments, the motor control portion is configured to actuate the first electro-mechanical actuator in response to a signal received from an external operator and to actuate the second electro-mechanical actuator in response to a signal produced by a sensor of the article. In some embodiments, the motor management portion is be configured to receive information from the signal management portion. For example, the motor management portion may be configured to receive signals from an external operator. In some embodiments, the motor management portion is configured to transmit information to the signal management portion (e.g., the motor management portion may be configured to transmit information regarding a state of the motors to the signal management portion) in order to transmit the information to the external operator.

In some embodiments, the circuit comprises a sensor management portion. The sensor management portion may be electrically connected to sensors of the article. The sensor management portion may receive signals from the sensors of the article. In some embodiments, the sensor management portion is configured to process signals from individual sensors (e.g., by consolidating signals from similar sensors to provide higher-resolution information). In some embodiments, the sensor processing circuit is configured to process information from the sensors (e.g., by filtering signal from the sensors). In some embodiments, the sensor management portion is configured to control the sensors (e.g., by turning the sensors off or on). The sensor management portion may be used to transmit information to the signal processing portion. For example, the sensor management portion may be used configured to transmit data from the sensor or sensors to an external operator.

In some embodiments, the circuit comprises a voltage management portion. The voltage management portion may comprise components constructed an arranged to amplify, modulate, or convert voltages associated with signals (e.g., external signals, or signals generated by sensors of the article) into voltages appropriate to actuate the article. The voltage management portion may be configured, for example, to output a uniform voltage to a motor control portion of an article in response to a continuously changing input received in the form of a signal from a sensor of the article. In some embodiments, the voltage management portion is electrically connected to a voltage source of the article. For example, the voltage management portion may be electrically connected to a battery of the article. In some embodiments, the article may comprise a power management portion, configured to supply power to an electro-mechanical actuator of the article. For example, the power management portion may comprise a super-capacitor and a voltage regulator electrically connected to the electro-mechanical actuator.

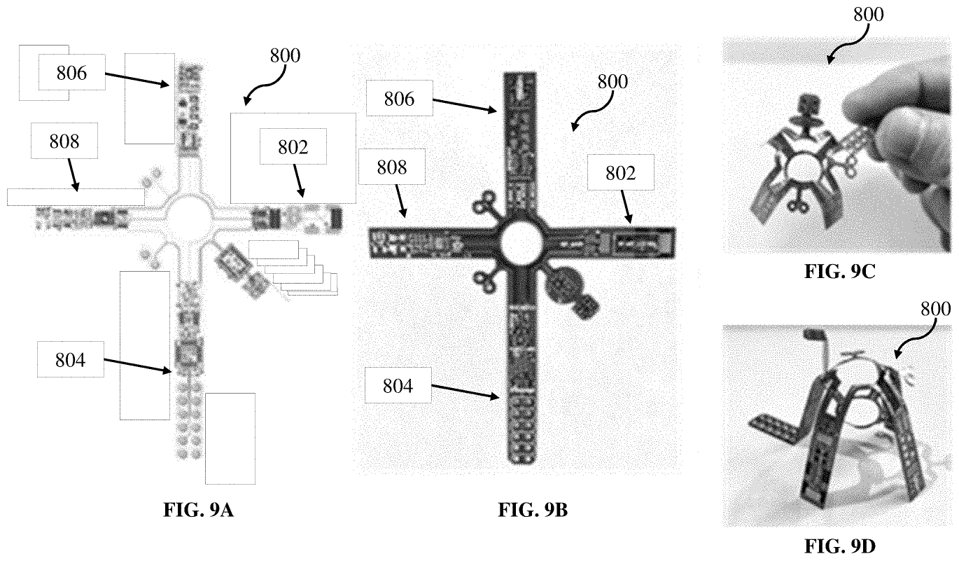

An exemplary circuit of an article is presented in FIGS. 9A-9D. FIG. 9A schematizes an exemplary circuit 800, which comprises signal processing portion 802, motor control portion 804, sensor control portion 806, and voltage management portion 808. FIG. 9B presents a photograph of circuit 800. In a planar configuration, while FIGS. 9C-9D present circuit 800 in a bent configuration appropriately shaped to be coupled to an article as described herein. A flexible circuit, such as the circuit shown in FIGS. 9B-9D, may be advantageous because the circuit can deform during changes in the state of the article, in some embodiments.

One advantage of the articles and circuits described herein, which has been inventively recognized in the context of the present disclosure, is that in some embodiments, the circuit can allow an article to perform closed-loop operations. In other words, the circuit can continuously update its operations based on input received from one or more sensors also located within the circuit. This may advantageously reduce or negate the need for an external operator, allowing the article to immediately respond to changes in its external condition. In some embodiments, the article described herein may administer an active pharmaceutical composition less than or equal to 10 minutes, less than or equal to 5 minutes, less than or equal to 1 minute, less than or equal to 30 seconds, or less after detection of radiation. In some embodiments, the article described herein may administer the active pharmaceutical composition less than or equal to 10 minutes, less than or equal to 5 minutes, less than or equal to 1 minute, less than or equal to 30 seconds, or less after detection of a chemical.

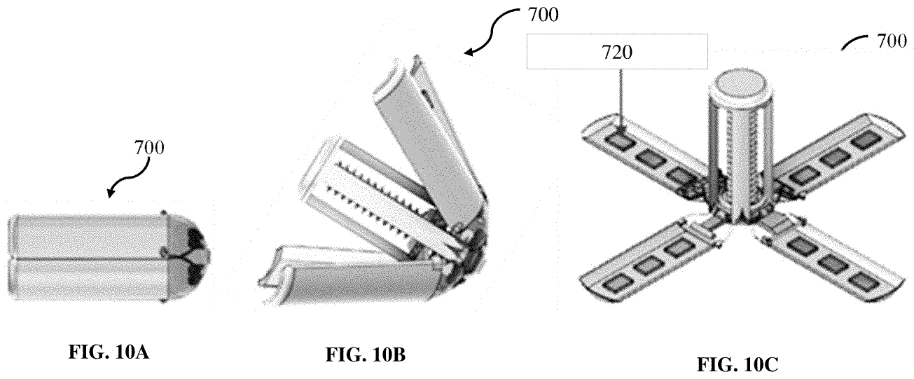

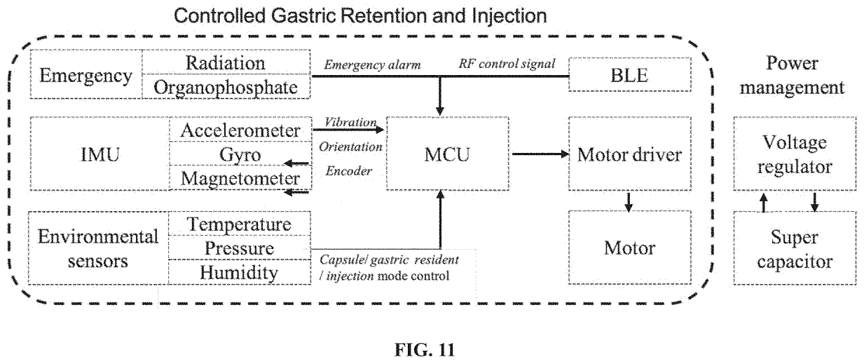

The article may comprise a sensor, in some embodiments. For example, the article may have one or multiple sensors, in some embodiments. The article has one or multiple radiation sensors, in some embodiments. The article has one or multiple chemical sensors, in some embodiments. According to certain embodiments, the article comprises multiple sensors of the same type. For example, the article may comprise multiple radiation sensors, in some embodiments. In some embodiments, the article comprises multiples sensors of different types. For example, the article may comprise a radiation sensor and a chemical sensor, in some embodiments. The sensors may be on the article. For example, the sensors may be on an arm of the article. In some embodiments, an arm of the article comprises one or more sensors. In some cases, each arm of the article comprises one or more sensors. In some embodiments, the central core comprises one or more sensors. An example is presented in FIGS. 10A-10C, which present article 700 transitioning from a retracted configuration (FIG. 10A) to an expanded configuration (FIG. 10C). In the expanded configuration (FIG. 10C), a plurality of sensors 720 are shown.

In some embodiments, the article comprises a radiation sensor. The radiation sensor may be configured to detect ionizing radiation. Examples of ionizing radiation include alpha, beta, gamma, neutron, proton, and heavy ion radiation. The radiation sensor may be any appropriate radiation sensor. For example, the radiation sensor may be configured to detect alpha radiation. The radiation sensor may be configured to detect beta radiation. In some embodiments, the radiation sensor is configured to detect gamma radiation. In some embodiments, the sensor detects radiation directly. For example, the sensor may comprise a semiconductor detector. In some embodiments, the sensor detects radiation indirectly. For instance, the article may comprise a scintillation detector, a photodetector, or an ionization chamber. In some embodiments, a radiation sensor comprises a photodetector comprising a photodiode. In some embodiments, a radiation detector comprises a plurality of photodiodes. In some embodiments, the photodiodes are PIN diodes. In some embodiments, the sensor comprises a volatile organic compound sensor incorporated in proximity to the photodiodes. According to certain embodiments, excitation of the volatile organic compound caused by ionizing radiation may produce light detected by the photodiodes. In the case of the exemplary embodiment of FIGS. 10A-10C, sensors 720 comprise PIN diode arrays configured to detect radiation. However, in principle, sensors 720 may be any of a variety of types of sensors, as described above.

In some embodiments, the article comprises a chemical sensor. The chemical sensor may be configured to detect a toxic chemical. For example, the chemical sensor may be configured to detect a biotoxin, a blood agent, an incapacitating agent, a metallic poison, a nerve agent, a toxic alcohol, a vomiting agent, and/or any other appropriate toxic chemical. The disclosure is not thus limited. Examples of biotoxins include, but are not limited to, abrin, anthrax toxin, brevetoxin, colchicine, digitalis, nicotine, ricin, saxitoxin, strychnine, tetrodotoxin, and trichothecene. Examples of blood agents include, but are not limited to, arsine, carbon monoxide, cyanide (e.g., cyanogen chloride, hydrogen cyanide, potassium cyanide and sodium cyanide). Examples of incapacitating agents include, but are not limited to, 3-Quinuclidinyl Benzilate (Agent BZ), and opioids (e.g., fentanyls). Examples of metallic poisons include, but are not limited to, arsenic, barium, mercury, and thallium. Examples of nerve agents include, but are not limited to, G agents, V agents, Novichok agents, carbamates, and insecticides.

In some embodiments, the chemical sensor is an organophosphate sensor. The organophosphate sensor may be configured to detect organophosphates. For example, the chemical sensor may be configured to detect organophosphates found in organophosphate nerve agents. Examples of organophosphate nerve agents include, but are not limited to, G agents, V agents, Novichok agents, and organophosphate insecticides. Examples of G agents include, but are not limited to, Sarin (GB), Soman (GD), Tabun (GA), Cyclosarin (GF), and GV. Examples of V agents include, but are not limited to, VX, VE, VG, VM, VR, VP, VS, EA-3148, and EA-2192. Examples of Novichok agents include, but are not limited to, Novichok-5 and Novichok-7. Examples of organophosphate insecticides include, but are not limited to, malathion, parathion, and chlorpyrifos.

The organophosphate sensor may operate according to any of a variety of principles known in the art. For example, the organophosphate sensor may comprise an organophosphate-sensitive hydrogel. The organophosphate-sensitive hydrogel may comprise oximes, in some embodiments. Without wishing to be bound by theory, according to certain embodiments oximes may react with organophosphate compounds, yielding phosphorylated adducts. Formation of these phosphorylated adducts can, in some embodiments, be optically detected by the sensor. For example, formation of these phosphorylated adducts may be detected by an absorption measurement, in some embodiments.

In some embodiments, the article comprises an organophosphate sensor.

According to certain embodiments, the article comprises spatial sensors. For example, the article may comprise an accelerometer. In some embodiments, the article comprises a gyro. In some embodiments, the article comprises a magnetometer. In some embodiments, signals from these sensors may be configured to provide information regarding the orientation and/or spatial position of the article.

In some embodiments, the article further comprises environmental sensors. Environmental sensors may detect environmental conditions external to the article. Environmental conditions external to the article may include temperature, pressure, humidity, and/or pH. In some embodiments, the article comprises a temperature sensor. In some embodiments, the article comprises a pressure sensor. In some embodiments, the article comprises a humidity sensor. In some embodiments, the article comprises a pH sensor. According to certain embodiments, environmental sensors may be used to determine the location of the article (e.g., within the gastrointestinal tract of a subject). Alternatively or additionally, environmental sensors may be used to monitor degradation of a capsule, e.g., a capsule comprising an article.

In some embodiments, one or more of the sensors of the article are operatively connected to the article. For example, the sensor may be operatively coupled to a drug release component of an article, in some embodiments. This may advantageously allow administration of an active pharmaceutical composition (e.g., comprising a counter-radiation agent) upon exposure to ionizing radiation above a threshold level. In some embodiments, this may allow administration of an active pharmaceutical composition (e.g., comprising a counter-toxin) upon exposure to toxins above a threshold level.

In some embodiments, the threshold level of ionizing radiation is a dose greater than or equal to 0.2 Gy, greater than or equal to 0.5 Gy, greater than or equal to 1 Gy, greater than or equal to 2 Gy, or greater. In some embodiments, the threshold level of ionizing radiation is a dose less than or equal to 10 Gy, less than or equal to 5 Gy, less than or equal to 2 Gy, less than or equal to 1 Gy, or less. Combinations of these ranges are possible. For example, in some embodiments, the threshold level of ionizing radiation is a dose greater than or equal to 0.2 Gy and less than or equal to 10 Gy. In some embodiments, a circuit may comprise a system architecture, in which information from sensors is provided to a processor, and in which the processor, in turn, actuates one or more electro-mechanical actuators (in this case, motors). FIG. 11 presents an exemplary such system architecture, wherein information from a radiation sensor, as well as positional and environmental sensors, is supplied to an processor (in this case, an MCU) configured to actuate one or more motors of the article. The system architecture further comprises a power management portion comprising a voltage regulator and a super capacitor, in order to provide power to the one or more motors, in some embodiments, as is shown in FIG. 11.

According to some embodiments, the articles described herein are compatible with one or more therapeutic, diagnostic, and/or enhancement agents, such as drugs, nutrients, microorganisms, in vivo sensors, and tracers. In some embodiments, the active substance, is a therapeutic, nutraceutical, prophylactic or diagnostic agent. While much of the specification describes the use of therapeutic agents, other agents listed herein are also possible.

For example, agents can include, but are not limited to, any synthetic or naturally-occurring biologically active compound or composition of matter which, when administered to a subject (e.g., a human or nonhuman animal), induces a desired pharmacologic, immunogenic, and/or physiologic effect by local and/or systemic action. For example, useful or potentially useful within the context of certain embodiments are compounds or chemicals traditionally regarded as drugs, vaccines, and biopharmaceuticals.

In some embodiments, the article comprises an active pharmaceutical composition (e.g., a therapeutic agent). The active pharmaceutical composition may be configured for release during internal residence of the article. For example, the active pharmaceutical agent may be configured for release at a location internal to a subject. In some embodiments, the active pharmaceutical composition is configured for release under a set of physiological conditions.

The active pharmaceutical composition may comprise one or more active pharmaceutical agents. The active pharmaceutical composition may have a solid form. For example, the active pharmaceutical composition may comprise one or more active pharmaceutical agents having a solid form. The active pharmaceutical composition may have a liquid form. For example, the active pharmaceutical composition may comprise a dissolved active pharmaceutical agent. The active pharmaceutical composition may be configured to be delivered via a drug release component as described herein.

Certain such agents may include molecules such as proteins, peptides, hormones, nucleic acids, gene constructs, etc., for use in therapeutic, diagnostic, and/or enhancement areas, including, but not limited to medical or veterinary treatment, prevention, diagnosis, and/or mitigation of disease or illness (e.g., HMG co-A reductase inhibitors (statins) like rosuvastatin, nonsteroidal anti-inflammatory drugs like meloxicam, selective serotonin reuptake inhibitors like escitalopram, blood thinning agents like clopidogrel, steroids like prednisone, antipsychotics like aripiprazole and risperidone, analgesics like buprenorphine, antagonists like naloxone, montelukast, and memantine, cardiac glycosides like digoxin, alpha blockers like tamsulosin, cholesterol absorption inhibitors like ezetimibe, metabolites like colchicine, antihistamines like loratadine and cetirizine, opioids like loperamide, proton-pump inhibitors like omeprazole, anti(retro)viral agents like entecavir, dolutegravir, rilpivirine, and cabotegravir, antibiotics like doxycycline, ciprofloxacin, and azithromycin, anti-malarial agents, and synthroid/levothyroxine); substance abuse treatment (e.g., methadone and varenicline); family planning (e.g., hormonal contraception); performance enhancement (e.g., stimulants like caffeine); and nutrition and supplements (e.g., protein, folic acid, calcium, iodine, iron, zinc, thiamine, niacin, vitamin C, vitamin D, and other vitamin or mineral supplements).

In an exemplary set of embodiments, the active substance is selected for treatment of acute radiation syndrome. In another exemplary set of embodiments, the active substance is selected for treatment of exposure to chemical toxins.

In certain embodiments, the active substance is one or more specific therapeutic agents. As used herein, the term “therapeutic agent” or also referred to as a “drug” refers to an agent that is administered to a subject to treat a disease, disorder, or other clinically recognized condition, or for prophylactic purposes, and has a clinically significant effect on the body of the subject to treat and/or prevent the disease, disorder, or condition. Listings of examples of known therapeutic agents can be found, for example, in the United States Pharmacopeia (USP), Goodman and Gilman's The Pharmacological Basis of Therapeutics, 10th Ed., McGraw Hill, 2001; Katzung, B. (ed.) Basic and Clinical Pharmacology, McGraw-Hill/Appleton & Lange; 8th edition (Sep. 21, 2000); Physician's Desk Reference (Thomson Publishing), and/or The Merck Manual of Diagnosis and Therapy, 17th ed. (1999), or the 18th ed (2006) following its publication, Mark H. Beers and Robert Berkow (eds.), Merck Publishing Group, or, in the case of animals, The Merck Veterinary Manual, 9th ed., Kahn, C. A. (ed.), Merck Publishing Group, 2005; and “Approved Drug Products with Therapeutic Equivalence and Evaluations,” published by the United States Food and Drug Administration (F.D.A.) (the “Orange Book”). Examples of drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. In certain embodiments, the therapeutic agent is a small molecule. Exemplary classes of therapeutic agents include, but are not limited to, analgesics, anti-analgesics, anti-inflammatory drugs, antipyretics, antidepressants, antiepileptics, antipsychotic agents, neuroprotective agents, anti-proliferatives, such as anti-cancer agents, antihistamines, antimigraine drugs, hormones, prostaglandins, antimicrobials (including antibiotics, antifungals, antivirals, antiparasitics), antimuscarinics, anxioltyics, bacteriostatics, immunosuppres sant agents, sedatives, hypnotics, antipsychotics, bronchodilators, anti-asthma drugs, cardiovascular drugs, anesthetics, anti-coagulants, inhibitors of an enzyme, steroidal agents, steroidal or non-steroidal anti-inflammatory agents, corticosteroids, dopaminergics, electrolytes, gastro-intestinal drugs, muscle relaxants, nutritional agents, vitamins, parasympathomimetics, stimulants, anorectics and anti-narcoleptics. Nutraceuticals can also be incorporated into the drug delivery article. These may be vitamins, supplements such as calcium or biotin, or natural ingredients such as plant extracts or phytohormones.

In another embodiment, the therapeutic agent is an immunosuppressive agent. Exemplary immunosuppressive agents include glucocorticoids, cytostatics (such as alkylating agents, antimetabolites, and cytotoxic antibodies), antibodies (such as those directed against T-cell receptors or Il-2 receptors), drugs acting on immunophilins (such as cyclosporine, tacrolimus, and sirolimus) and other drugs (such as interferons, opioids, TNF binding proteins, mycophenolate, and other small molecules such as fingolimod).

In some embodiments, the therapeutic agent is a small molecule drug having molecular weight less than about 2500 Daltons, less than about 2000 Daltons, less than about 1500 Daltons, less than about 1000 Daltons, less than about 750 Daltons, less than about 500 Daltons, less or than about 400 Daltons. In some cases, the therapeutic agent is a small molecule drug having molecular weight between 200 Daltons and 400 Daltons, between 400 Daltons and 1000 Daltons, or between 500 Daltons and 2500 Daltons.

In certain embodiments, the therapeutic agent is present in the article in an amount greater than or equal to 1 gram, greater than or equal to 2 grams, greater than or equal to 3 grams, greater than or equal to 5 grams, greater than or equal to 10 grams, greater than or equal to 20 grams, greater than or equal to 30 grams, greater than or equal to 40 grams, greater than or equal to 50 grams, greater than or equal to 60 grams, greater than or equal to 70 grams, or greater than or equal to 80 grams, greater than or equal to 90 grams. In some embodiments, the therapeutic agent is present in the article in an amount of less than or equal to 100 grams, less than or equal to 90 grams, less than or equal to 80 grams, less than or equal to 70 grams, less than or equal to 60 grams, less than or equal to 50 grams, less than or equal to 40 grams, less than or equal to 30 grams, less than or equal to 20 grams, less than or equal to 10 grams, less than or equal to 5 grams, less than or equal to 3 grams, or less than or equal to 2 grams. Combinations of the above-referenced ranges are also possible (e.g., greater than or equal to 1 gram and less than or equal to 100 grams, greater than or equal to 2 grams and less than or equal to 100 grams, greater than or equal to 3 grams and less than or equal to 100 grams). Other ranges are also possible.

In some embodiments, the articles described herein comprises two or more types of therapeutic agents. For example, in some embodiments, a first therapeutic agent and a second therapeutic agent are present in the article such that the total amount of the first and second therapeutic agent is in one or more ranges described above (e.g., the total amount of therapeutic agent is greater than or equal to 1 gram and less than or equal to 100 grams). In some embodiments, each therapeutic agent is present in an amount such that the total amount of therapeutic agents is greater than or equal to 1 gram. In some embodiments, each therapeutic agent is present in an amount as described above (e.g., each therapeutic agent is present in an amount of greater than or equal to 1 gram and less than or equal to 100 grams).

In certain embodiments, the therapeutic agent is present in the article at a concentration such that, upon release from the article, the therapeutic agent elicits a therapeutic response.

In some embodiments, a subject may demonstrate health benefits, e.g., upon administration of the article (e.g., treatment for radiation exposure, treatment for chemical toxin exposure).

The active pharmaceutical agent may comprise a counter-radiation agent, in some embodiments. The counter-radiation agent comprises a chelating agent, in some embodiments. The counter-radiation agent comprises a salt, in some embodiments. In some embodiments, the counter-radiation agent comprises iodine. According to certain embodiments, the counter-radiation agent comprises a scavenger for reactive oxygen species (an ROS scavenger). In some embodiments, the counter-radiation agent comprises an active pharmaceutical agent appropriate for treating symptoms associated with radiation exposure. The active pharmaceutical composition may comprise more than one counter-radiation agent. For example, the active pharmaceutical composition may comprise one or more counter-radiation agents configured to absorb radiation, as well as one or more counter-radiation agents configured to treat symptoms associated with radiation exposure. It should, of course, be understood that these examples are non-limiting and that a counter-radiation agent may comprise any active pharmaceutical agent indicated for the treatment of exposure to radiation (e.g., ionizing radiation).

In some embodiments, the counter-radiation agent comprises chelating agent. For example, in some embodiments, the counter-radiation agent is a chelating agent. The counter-radiation agent may be configured to bind chelate radioactive ions. In some embodiments, chelated ions may be excreted from the body along with the chelating agent, advantageously reducing exposure to the chelated ions. The counter-radiation agent may be configured to chelate cesium, thallium, uranyl, americium, curium, plutonium, or iodine ions. In some embodiments, the chelating agent comprises Prussian blue. Without wishing to be bound by theory, Prussian blue may chelate radioactive cesium and/or thallium ions. In some embodiments, the chelating agent comprises diethylenetriamine pentaacetic acid (DTPA). Without wishing to be bound by theory, DTPA may chelate radioactive elements such as plutonium, americium, and curium.

In some embodiments, the counter-radiation agent comprises a salt. For example, the counter-radiation agent may be a salt, in some embodiments. The salt may be selected to displace radioactive materials that bind to portions of the body. For example, the salt may be an iodide salt. Without wishing to be bound by theory, in some embodiments, the iodide salt may be used to displace radioactive iodine absorbed by the thyroid, in some embodiments. In some embodiments, the iodide salt may comprise sodium iodide or potassium iodide. In some embodiments, the counter-radiation agent comprises pure iodine (I₂). Similarly to iodine salts, without wishing to be bound by theory, pure iodine may be used to displace radioactive iodine absorbed by the thyroid, in some embodiments.

In some embodiments, the counter-radiation agent comprises a reactive oxygen species (ROS) scavenger. The ROS scavenger may be configured to bind to reactive oxygen species in a subject. For example, the ROS scavenger may be configured to bind to reactive oxygen species produced by exposure to ionizing radiation. In some embodiments, the ROS scavenger comprises amifostene and/or a superoxide dismutase mimetic (e.g., avasopasem manganese).

According to certain embodiments, the counter-radiation agent comprises an active pharmaceutical agent appropriate for treating symptoms associated with radiation exposure. Exemplary symptoms of radiation exposure include but are not limited to: bacterial infections, headache, inflammation, fever, diarrhea, nausea, vomiting, dehydration, burns, sores, neurological damage, and ulcers. In some embodiments, the counter-radiation agent comprises an antimicrobial agent. For example, the counter radiation agent may comprise an antibiotic or an antifungal medication. In some embodiments, the counter-radiation agent comprises an anti-inflammatory agent. In some embodiments, the counter radiation agent may comprise a growth factor. As an example, the counter radiation agent may comprise a growth factor comprising sargramostim. In some embodiments, the counter-radiation agent comprises an antinausea agent. Without wishing to be bound by theory, in some embodiments, inclusion of a counter-radiation agent comprising an active pharmaceutical agent appropriate for treating symptoms associated with radiation exposure may be advantageous, for example, for treatment of symptoms associated with radiation therapies.

The active pharmaceutical composition may comprise a counter-toxin, in some-embodiments. The counter-toxin may comprise an antidote for a toxin, according to certain embodiments. For example, the counter-toxin may comprise an antitoxin, in some embodiments. According to certain embodiments, the counter agent comprises a competitive inhibitor. In some embodiments, the counter-toxin comprises a chelator. According to certain embodiments, the counter-toxin comprises an active pharmaceutical agent appropriate for treating symptoms associated with exposure to a toxin. It should, of course, be understood that these examples are non-limiting and that a counter-toxin may comprise any active pharmaceutical agent indicated for the treatment of exposure to a toxin detected by a sensor, as described above. According to certain embodiments, the counter-toxin is specific to a particular toxin or class of toxins.

The counter-toxin may be a nerve agent counter-toxin. In some embodiments, the counter-toxin is configured to bind to a nerve agent. For instance, the counter-toxin may comprise one or more types of oxime, suitable for binding the nerve agent. For example, the counter-toxin may comprise pralidoxime chloride. Without wishing to be bound by theory, the oxime may result in detachment of the nerve agent from cholinesterase enzymes, reactivating the cholinesterase enzymes. In some embodiments, the counter-toxin is a competitive inhibitor to a nerve agent. For example, the nerve agent counter-toxin may comprise atropine. Without wishing to be bound by theory, atropine may advantageously reduce the effect of excessive acetylcholine action, an undesirable symptom of exposure to the nerve agent, by reversibly binding to cholinesterase enzymes. In some embodiments, the nerve agent counter-toxin comprises an active pharmaceutical agent appropriate for treating symptoms associated with exposure to a nerve agent.

Active pharmaceutical compositions for use in accordance with the present disclosure may include a pharmaceutically acceptable excipient. As used herein, the term “pharmaceutically acceptable excipient” means a non-toxic, inert solid, semi-solid or liquid filler, diluent, encapsulating material or formulation auxiliary of any type. Some examples of materials which can serve as pharmaceutically acceptable excipients are sugars such as lactose, glucose, and sucrose; starches such as corn starch and potato starch; cellulose and its derivatives such as sodium carboxymethyl cellulose, methylcellulose, hydroxypropylmethylcellulose, ethyl cellulose, and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as Tween 80; buffering agents such as magnesium hydroxide and aluminum hydroxide; alginic acid; pyrogen free water; isotonic saline; citric acid, acetate salts, Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension, or emulsion in a nontoxic parenterally acceptable diluent or solvent, for example, as a solution in 1,3-butanediol. Among the acceptable vehicles and solvents that may be employed are water, Ringer's solution, ethanol, U.S.P., and isotonic sodium chloride solution. In addition, sterile, fixed oils are conventionally employed as a solvent or suspending medium. For this purpose, any bland fixed oil can be employed including synthetic mono or diglycerides. In addition, fatty acids such as oleic acid are used in the preparation of injectables.

The injectable formulations can be sterilized, for example, by filtration through a bacteria retaining filter, or by incorporating sterilizing agents in the form of sterile solid compositions which can be dissolved or dispersed in sterile water or other sterile injectable medium prior to use.

In some embodiments, the article may be configured to adjust various parameters based on physiological and/or external metrics. For example, in some embodiments, the article is configured to adjust the rate and/or amount of a pharmaceutical agent released from the article (e.g., stored within one or more reservoirs associated with the article) e.g., in response to a signal from a sensor in electrical or wireless communication with and/or associated with (e.g., embedded within) the article. In some embodiments, the article adjusts the rate and/or amount of a pharmaceutical agent released from the article in response to an input from the user and/or a signal from the sensor. In some embodiments, the article is associated with one or more reservoirs configured for the release of a pharmaceutical agent. In some embodiments, the one or more reservoirs may release a portion of the pharmaceutical agent contained therein in response to a signal received from a sensor in electrical or wireless communication with the article.

In some embodiments, the article is configured to adjust the position and/or orientation of the article (e.g., via the one or more arms) e.g., in response to a signal from a sensor in electrical or wireless communication with and/or associated with (e.g., embedded within) the article.

Non-limiting examples of suitable sensors for use with the structures and methods described herein include radiation sensors, chemical toxin sensors, temperature sensors (e.g., monitoring internal temperature, ambient temperature, temperature of a component associated with the article such as a thermally sensitive polymer), physiological/biometric sensors (e.g., heart rate, electrical activity, neuronal activity), accelerometers (e.g., for measuring breathing rate, activity levels, sleeping behavior/patterns, article orientation), and environmental sensors (e.g., pH, biologic concentration, chemical concentration).

Any article circuitry may be implemented by any suitable type of analog and/or digital circuitry. For example, the article circuitry may be implemented using hardware or a combination of hardware and software. When implemented using software, suitable software code can be executed on any suitable processor (e.g., a microprocessor) or collection of processors. The one or more articles can be implemented in numerous ways, such as with dedicated hardware, or with general purpose hardware (e.g., one or more processors) that is programmed using microcode or software to perform the functions recited above.

The above-described embodiments of the technology described herein can be implemented in any of numerous ways. For example, the embodiments may be implemented using hardware, software or a combination thereof. When implemented in software, the software code can be executed on any suitable processor or collection of processors, whether provided in a single computing device or distributed among multiple computing devices. Such processors may be implemented as integrated circuits, with one or more processors in an integrated circuit component, including commercially available integrated circuit components known in the art by names such as CPU chips, GPU chips, MPU chips, microprocessor, microcontroller, or co-processor. Alternatively, a processor may be implemented in custom circuitry, such as an ASIC, or semicustom circuitry resulting from configuring a programmable logic device. As yet a further alternative, a processor may be a portion of a larger circuit or semiconductor device, whether commercially available, semi-custom or custom. As a specific example, some commercially available microprocessors have multiple cores such that one or a subset of those cores may constitute a processor. Though, a processor may be implemented using circuitry in any suitable format.

Further, it should be appreciated that a computing device may be embodied in any of a number of forms, such as a rack-mounted computer, a desktop computer, a laptop computer, or a tablet computer. Additionally, a computing device may be embedded in a device not generally regarded as a computing device but with suitable processing capabilities, including a Personal Digital Assistant (PDA), a smart phone, tablet, or any other suitable portable or fixed electronic device.

Also, a computing device may have one or more input and output devices. These devices can be used, among other things, to present a user interface. Examples of output devices that can be used to provide a user interface include display screens for visual presentation of output and speakers or other sound generating devices for audible presentation of output. Examples of input devices that can be used for a user interface include keyboards, individual buttons, and pointing devices, such as mice, touch pads, and digitizing tablets. As another example, a computing device may receive input information through speech recognition or in other audible format.

Such computing devices may be interconnected by one or more networks in any suitable form, including as a local area network or a wide area network, such as an enterprise network or the Internet. Such networks may be based on any suitable technology and may operate according to any suitable protocol and may include wireless networks, wired networks or fiber optic networks. For example, in some embodiments, the article comprises wireless capabilities for enabling suitable communication with other devices/systems (e.g., for controlling aspects of the article, controlling/monitoring physiological conditions of the subject (e.g., at the location internal to the subject), etc.). Wireless devices are generally known in the art and may include, in some cases, LTE, WiFi and/or Bluetooth systems. In some embodiments, the articles described herein comprise such a wireless device.

Also, the various methods or processes outlined herein may be coded as software that is executable on one or more processors that employ any one of a variety of operating systems or platforms. Additionally, such software may be written using any of a number of suitable programming languages and/or programming or scripting tools, and also may be compiled as executable machine language code or intermediate code that is executed on a framework or virtual machine.

In this respect, the embodiments described herein may be embodied as a computer readable storage medium (or multiple computer readable media) (e.g., a computer memory, one or more floppy discs, compact discs (CD), optical discs, digital video disks (DVD), magnetic tapes, flash memories, RAM, ROM, EEPROM, circuit configurations in Field Programmable Gate Arrays or other semiconductor devices, or other tangible computer storage medium) encoded with one or more programs that, when executed on one or more computers or other processors, perform methods that implement the various embodiments discussed above. As is apparent from the foregoing examples, a computer readable storage medium may retain information for a sufficient time to provide computer-executable instructions in a non-transitory form. Such a computer readable storage medium or media can be transportable, such that the program or programs stored thereon can be loaded onto one or more different computing devices or other processors to implement various aspects of the present disclosure as discussed above. As used herein, the term “computer-readable storage medium” encompasses only a non-transitory computer-readable medium that can be considered to be a manufacture (i.e., article of manufacture) or a machine. Alternatively or additionally, the disclosure may be embodied as a computer readable medium other than a computer-readable storage medium, such as a propagating signal.

The terms “program” or “software” are used herein in a generic sense to refer to any type of computer code or set of computer-executable instructions that can be employed to program a computing device or other processor to implement various aspects of the present disclosure as discussed above. Additionally, it should be appreciated that according to one aspect of this embodiment, one or more computer programs that when executed perform methods of the present disclosure need not reside on a single computing device or processor, but may be distributed in a modular fashion amongst a number of different computers or processors to implement various aspects of the present disclosure.

Computer-executable instructions may be in many forms, such as program modules, executed by one or more computers or other devices. Generally, program modules include routines, programs, objects, components, data structures, etc. that perform particular tasks or implement particular abstract data types. Typically the functionality of the program modules may be combined or distributed as desired in various embodiments.

The embodiments described herein may be embodied as a method, of which an example has been provided. The acts performed as part of the method may be ordered in any suitable way. Accordingly, embodiments may be constructed in which acts are performed in an order different than illustrated, which may include performing some acts simultaneously, even though shown as sequential acts in illustrative embodiments.

Further, some actions are described as taken by a “user.” It should be appreciated that a “user” need not be a single individual, and that in some embodiments, actions attributable to a “user” may be performed by a team of individuals and/or an individual in combination with computer-assisted tools or other mechanisms.

The following examples are intended to illustrate certain embodiments of the present invention, but do not exemplify the full scope of the invention.

### EXAMPLE 1

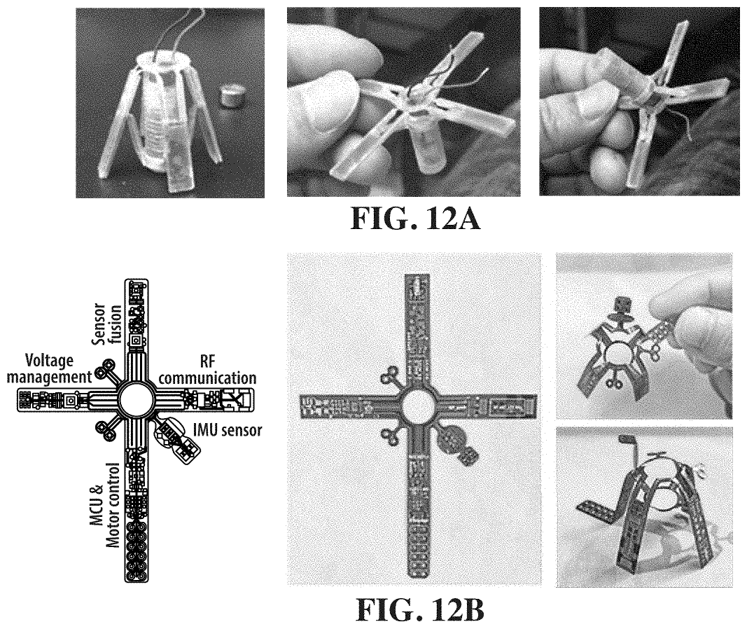

FIGS. 12A-12B present an exemplary implementation of an article having deployable arms (FIG. 12A) and a Kirigami circuit design contained on the deployable arms (FIG. 12B). FIGS. 9A-9D present the circuit in greater detail. In the exemplary embodiment of FIGS. 12A-12B, the deployable arms are configured to allow long residential times in the stomach, as well as self-orientation for actuation of drug delivery into the correct location.

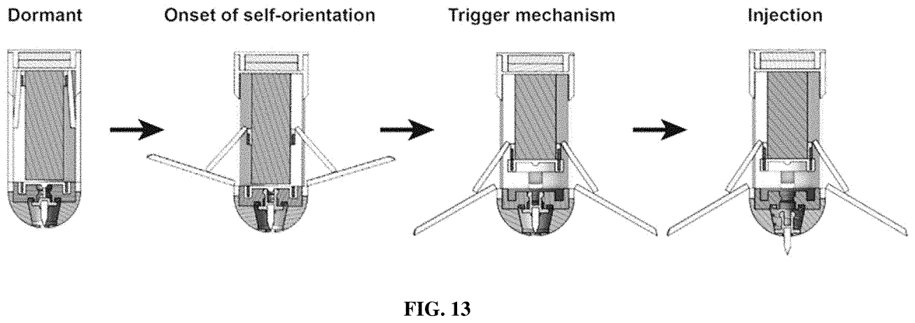

FIG. 4, described in greater detail above, illustrates the article during controlled gastric residence. The exemplary article of FIGS. 12A-12B is configured to detect radiation and chemical exposure in the core body area and deliver the medicine upon the detection. The article could control its orientation to inject active pharmaceutical agents into stomach tissue. For the alpha, beta, and gamma radiation sensor, a PIN diode with varying number of photodiodes was used. Volatile organic compound sensors were incorporated in proximity of the photodiodes. FIGS. 10A-10C, described above, and FIG. 13 illustrate computer aided design (CAD) schematics of the controlled orientation of the article for sensing and patrolling, as well as injection of the active pharmaceutical ingredient (API) into the gastric wall upon exposure to threshold dose of radiation or chemical.

This example demonstrates that a functional article has been successfully prepared that incorporates many of the features described above.

### EXAMPLE 2

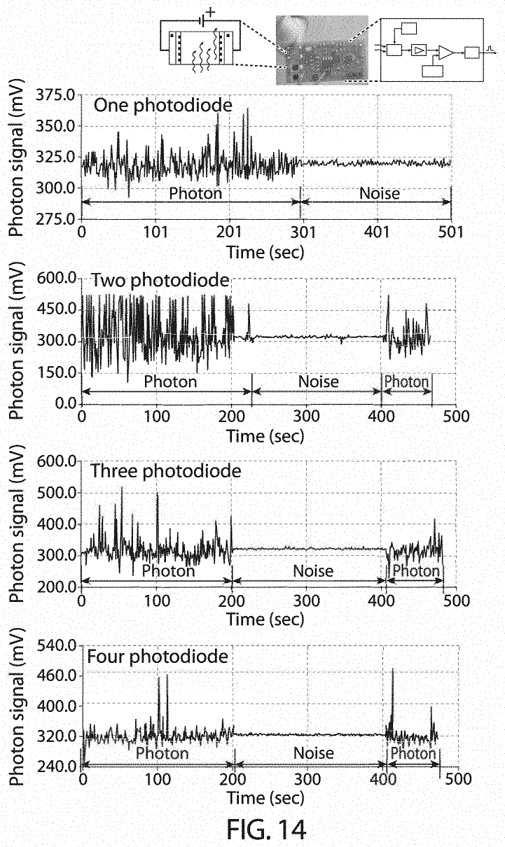

Photon signal from the article of Example 1 can be found in FIG. 14. FIG. 14 presents the signal detected by the article using one photodiode, two photodiodes, three photodiodes, and four photodiodes. As illustrated, the article is capable of detecting photon signal exceeding noise, and is therefore capable of detecting photon emissions from volatile organic compounds, thereby allowing detection of radioactive materials.

### EXAMPLE 3

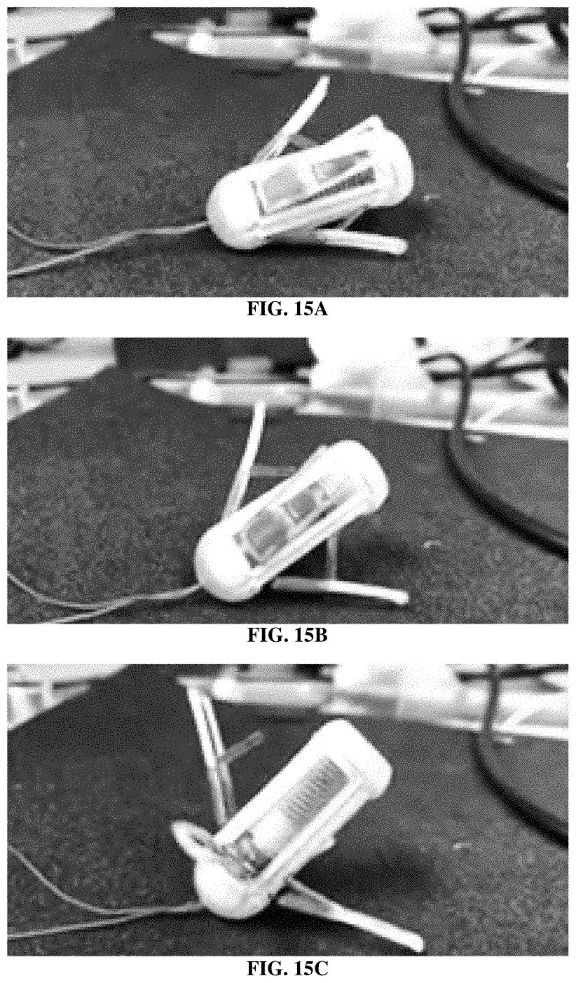

This example demonstrates self-righting of an exemplary prototype of an article analogous to the article represented in FIGS. 2A-2C, differing from the exemplary that the article does not comprise a battery, drug release component, drug reservoir component, anchoring component or locking mechanism. Instead, the article comprises wire connections to a power supply were passed through a prototypical tissue-interfacing surface to power an electro-mechanical actuator (in this case, a motor) connected to the rigid body of the exemplary article. In this example, the article demonstrates self-righting via the transition of the article to a more expanded configuration from a retracted configuration. Video of the process was recorded, and select moments of the process are shown in FIGS. 15A-15C. In FIG. 15A, the article is shown in a retracted configuration, resting on a surface. Once the motor was actuated, the article began to rotate, resting on the two of its arms that sat on the surface, while the central core of the article became oriented in a more vertical direction. The partially expanded configuration of the article is shown in FIG. 15B. Finally, the article reached an even more vertical orientation of the central core, as shown in FIG. 15C.

The article of this embodiment was unable to reach a perfectly vertical orientation, since the wires passing through the tissue-interfacing surface prevented its continued rotation, this example demonstrates that the self-righting mechanism described herein is viable for articles comprising arms and central cores.

### EXAMPLE 4

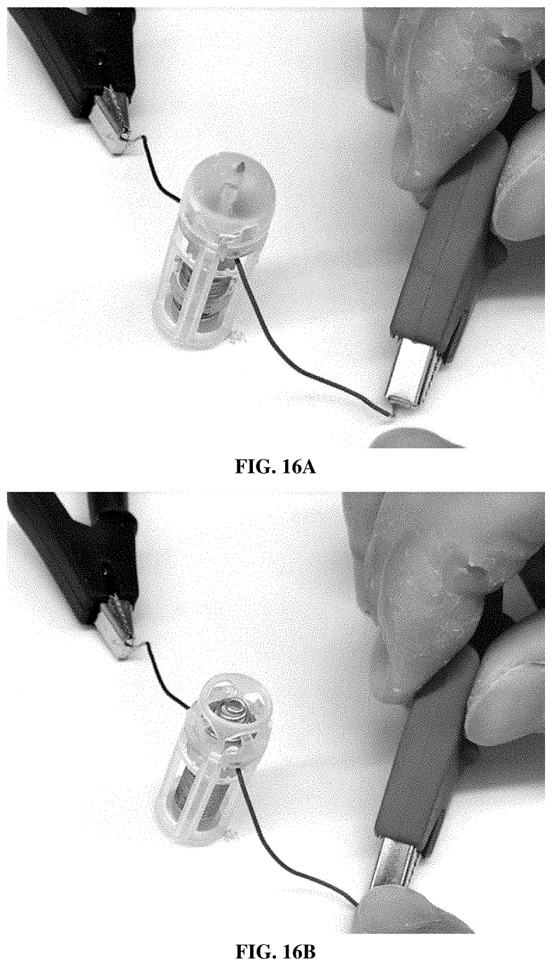

This example demonstrates the actuation of a tissue interfacing article comprising an anchoring component and a locking mechanism, as described herein. Initially, the drug reservoir component of the article, coupled to the drug release component of the article, was held inside the exemplary article, shown in a first configuration of the article in FIG. 16A. However, by supplying voltage to actuate the electro-mechanical actuator coupled to the locking mechanism, the locking mechanism was removed from contact with the anchoring component. Once the locking mechanism was removed from the anchoring component, the spring ejected the drug reservoir component from the article, launching the tissue-interfacing surface of the article, the drug reservoir component and the drug release component away from the central core of the article, leaving the spring visible, as shown in FIG. 16B. This example demonstrates the viability of this construction for delivery of drug release components and drug reservoir components to a subject.

### EXAMPLE 5

This example demonstrates the delivery of active pharmaceutical compositions using an exemplary drug release component in vivo. The exemplary drug release component was incorporated into a self-orienting millimeter scale applicator (SOMA) articles and administered to a subject in vivo.

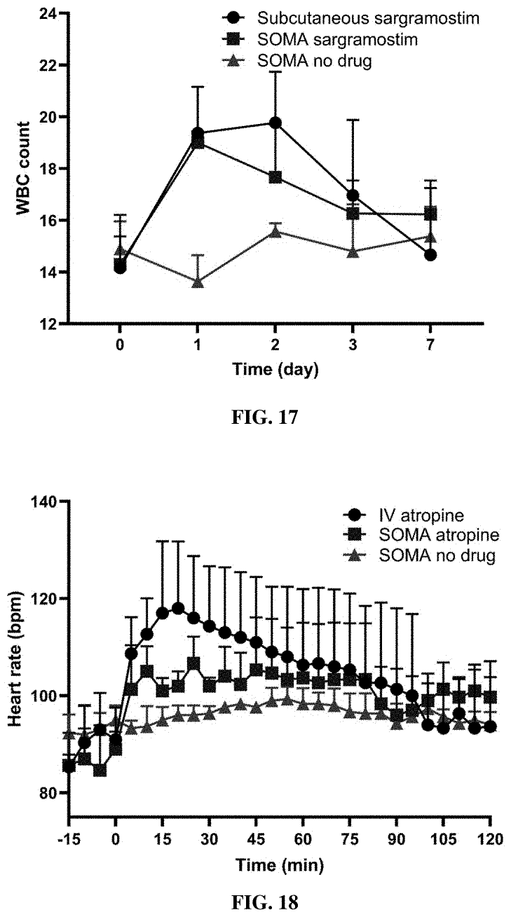

In one experiment, a counter-radiation agent, sargramostim, which is used to increase white blood cell counts (WBC counts) after acute radiation exposure, was administered to subjects. Experiments were performed in Yorkshire pigs (45-70 kg) (n=3-4). Baseline CBCs (complete blood counts) were obtained prior to administration of drug. A single 250 microgram dose was administered, using the exemplary drug release component, into the gastric wall of anesthetized pigs through an endoscopically placed SOMA article. CBCs were obtained daily for 7 days after administration of the sargramostim. The white blood cell counts of these animals were compared to separate control cohorts of pigs that were given either a single 250 microgram subcutaneous dose of sargramostim (clinical standard-of-care) or endoscopically placed non-drug loaded SOMA articles. The effect of this dose on white blood count was compared with a dose of sagramostim administered subcutaneously, and with measurements taken from a control sample, wherein the exemplary drug release component did not comprise the active pharmaceutical agent. The results are presented in FIG. 17, which shows the WBC count over time as a function of each dosing condition. This result indicates that delivery of the sargramostim using the exemplary drug release component was effective, although the WBC count was not identical to a dose administered subcutaneously.

In another experiment, a counter-toxin, atropine, was delivered using the exemplary drug release component. Atropine can be monitored via its effect on heart rate. Experiments were performed in Yorkshire pigs (45-70 kg) (n=3-4). Baseline vital signs were obtained for 30 minutes prior to administration of drug. A single 3 mg dose was administered into the gastric wall of anesthetized pigs through an endoscopically placed SOMA article. Vital signs were monitored for 2 hours after administration of the atropine. The white blood cell counts of these animals were compared to separate control cohorts of pigs that were given either a single 3 mg intravenous dose of atropine (clinical standard-of-care) or endoscopically placed non-drug loaded SOMA articles. Although the heart rate of subjects receiving IV atropine initially exceeded the heart rate of subjects receiving atropine via a drug release component, recipients of atropine from the drug release component still experienced an increased heart rate, relative to the control group, indicating that the atropine remained effective when delivered in this fashion. Furthermore, the effects of the atropine delivered via the drug release component lasted longer, indicating that delivery using a drug release component may advantageously act as a mechanism for control release of atropine.

These experiments demonstrate the viability of exemplary drug release components for administration of active pharmaceutical compositions to subjects.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, and/or method described herein. In addition, any combination of two or more such features, systems, articles, materials, and/or methods, if such features, systems, articles, materials, and/or methods are not mutually inconsistent, is included within the scope of the present invention.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified unless clearly indicated to the contrary. Thus, as a non-limiting example, a reference to “A and/or B,” when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

As used herein, “wt %” is an abbreviation of weight percentage. As used herein, “at %” is an abbreviation of atomic percentage.

Some embodiments may be embodied as a method, of which various examples have been described. The acts performed as part of the methods may be ordered in any suitable way. Accordingly, embodiments may be constructed in which acts are performed in an order different than illustrated, which may include different (e.g., more or less) acts than those that are described, and/or that may involve performing some acts simultaneously, even though the acts are shown as being performed sequentially in the embodiments specifically described above.

Use of ordinal terms such as “first,” “second,” “third,” etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements.

In the claims, as well as in the specification above, all transitional phrases such as “comprising,” “including,” “carrying,” “having,” “containing,” “involving,” “holding,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03.

1-15. (canceled)

16. An article, comprising:
a central core,
a tissue-engaging surface,
a plurality of arms connected to the central core,
one or more radiation sensors on the plurality of arms, and
a drug releasing component associated with the tissue-engaging surface.

17. The article of claim 16, further comprising an active pharmaceutical composition.

18. The article of claim 16, having an expanded configuration and a retracted configuration.

19. The article of claim 16, wherein the article further comprises one or more elastic components connected to the plurality of arms and configured to bias the arms away from the central core, and one or more biasing components connected to the plurality of arms and configured to bias the arms towards the central core.

20. The article of claim 16, wherein the article further comprises:
a rigid body;
an arm connector coupled to the rigid body at a proximal end of the arm connector and coupled to an arm of the plurality of arms at a distal end of the arm connector; and
an electro-mechanical actuator configured to translate the rigid body in a direction parallel to the central axis;
wherein translation of the rigid body in the direction parallel to the central axis changes the configuration of the article between the expanded configuration and the retracted configuration.

21. The article of claim 16, wherein in the retracted configuration, the article may be ingested.

22. The article of claim 16, wherein the active pharmaceutical composition comprises a counter-radiation agent.

23. The article of claim 16, wherein the active pharmaceutical composition is a counter-radiation agent.

24. The article of claim 16, wherein the active pharmaceutical composition comprises an iodine salt, a chelating agent, and/or a reactive oxygen species scavenger.

25. The article of claim 16, wherein the radiation sensor is configured to detect ionizing radiation.

26. The article of claim 25, wherein the ionizing radiation comprises alpha, beta, gamma, neutron, proton, and/or heavy ion radiation.

27-29. (canceled)

30. The article of claim 16, wherein upon detection of radiation above a threshold level, the article is configured to deliver the active pharmaceutical composition agent using the drug releasing component.

31. The article of claim 16, wherein the drug releasing component comprises a needle.

32. The article of claim 16, wherein the article is self-righting.

33-34. (canceled)

35. A method, comprising:
detecting radiation using a radiation sensor of an article located within a subject; and
upon detection of radiation above a threshold level, administering an active pharmaceutical agent to the subject using the article.

36-39. (canceled)

40. The method of claim 35, further comprising retaining the article in a stomach until radiation above a threshold level is detected.

41-45. (canceled)

46. An article, comprising:
a central core,
a tissue-engaging surface,
a plurality of arms connected to the central core,
one or more chemical sensors on the plurality of arms, wherein the one or more sensors are configured to detect a toxic chemical, and
a drug releasing component associated with the tissue-engaging surface.

47. The article of claim 46, further comprising an active pharmaceutical composition.

48-53. (canceled)

54. The article of claim 46, wherein the chemical sensor is configured to detect a nerve agent.

55. (canceled)

56. The article of claim 46, wherein the article comprises the active pharmaceutical agent appropriate for treating symptoms associated with exposure to a nerve agent, and wherein the active pharmaceutical agent comprises atropine.

57. The article of claim 46, wherein upon detection of a toxin, the article is configured to deliver the active pharmaceutical composition agent using the drug releasing component.

58. The article of claim 46, wherein the drug releasing component comprises a needle.

59. The article of claim 46, wherein the article is self-righting.

60-70. (canceled)
